(19) United States
(12) Patent Application Publication
(10) Pub. No.: US 2015/0147384 A1
(43) Pub. Date: May 28, 2015

(54) Sustained Delivery of Molecules Using Peptide Surfactants and Uses Thereof

(71) Applicant: Massachusetts Institute of Technology, Cambridge, MA (US)

(72) Inventors: Sotirios Koutsopoulos, Boston, MA (US); Shuguang Zhang, Lexington, MA (US)

(21) Appl. No.: 14/546,454

(22) Filed: Nov. 18, 2014

Related U.S. Application Data
(63) Continuation of application No. PCT/US13/43572, filed on May 31, 2013.
(60) Provisional application No. 61/653,782, filed on May 31, 2012.

(51) Int. Cl. A61K 9/51; A61K 31/7088

(52) U.S. Cl. CPC A61K 9/5123; A61K 31/7088
USPC 424/450; 514/44 R; 424/489

(57) ABSTRACT

The present invention is directed to a drug delivery composition comprising a self-assembled nanostructure and biologically active molecule, wherein the nanostructure comprises surfactant peptides, and wherein the biologically active molecule is encapsulated in the nanostructure. The invention also encompasses methods of administering a biologically active molecule to a subject comprising administering the drug delivery composition and methods for the preparation of said composition.

### RELATED APPLICATION

This application is a continuation of International application No.: PCT/US13/43572, which designated the United States and was filed on May 31, 2013, published in English which claims the benefit of U.S. Provisional Application No. 61/653,782, filed May 31, 2012. The entire teachings of the above applications are incorporated herein by reference.

### BACKGROUND OF THE INVENTION

Molecular self-assembly has led to the fabrication of nanostructures and the development of advanced materials [1]. Molecular design and synthesis of small, biologically inspired biomolecules including lipids, peptides, oligonucleotides, and polysaccharides with self-assembling properties has significantly advanced the field of biomaterials [2]. Short peptides with self-assembling properties were discovered in 1993 and since then a variety of amino acid sequences have been synthesized. Depending on the sequence, self-assembling peptides have varying properties and have been tested for applications in biomedicine as permissive biological scaffolds for regenerative medicine and drug delivery systems [3,4,5]. Self-assembly of amphiphilic peptides leads to the formation of stable nanotubes, vesicles or micelles similar to lipids and other chemical surfactants [6].

The type and size of the resulting peptide assembly ultra-structures e.g., nanotubes, nanodoughnuts, nanovalves, nanovesicles, or micelles, depend on the peptide concentration, the peptide's critical micelle concentration (CMC), the amino acid sequence, geometrical constraints (which are defined by the length of the side groups of the amino acids), the type and charge of electrolyte used to induce self-assembly, the ionic strength and pH of the medium [6, 7, 8, 9]. These factors affect peptide alignment, the packing density, and the strength of the inter-molecular bonds between the monomers which in turn result in hierarchical supramolecular structures of different morphologies and properties [6, 7]. The development of self-assembling peptides with amphiphilic properties have opened new avenues for applications in biotechnology for the stabilization of membrane proteins more effectively than commercial detergents [10,11] and in nanotechnology for the construction of energy conversion devices [12].

### SUMMARY OF THE INVENTION

The present invention is directed to an additional application for self-assembling peptides. The invention is directed to a drug delivery composition comprising a self-assembled nanostructure and biologically active molecule, wherein the nanostructure comprises surfactant peptides, and wherein the biologically active molecule is encapsulated in the nanostructure. The invention also encompasses methods of administering a biologically active molecule to a subject comprising administering a drug delivery composition described herein and methods for the preparation of the drug delivery composition.

The invention encompasses a drug delivery composition comprising a self-assembled nanostructure and a biologically active molecule, wherein the nanostructure comprises a plurality of surfactant peptides and wherein the surfactant peptides have a formula selected from the group consisting of:

a. (Φ)ₘ(+)ₙ (Formula (1)),

b. (+)ₙ(Φ)ₘ (Formula (2)),

c. (Φ)ₘ(−)ₙ (Formula (3)),

d. (−)ₙ(Φ)ₘ (Formula (4)),

e. (−)ₙ(Φ)ₘ(−)ₙ (Formula (5)),

f. (+)ₙ(Φ)ₘ(+)ₙ (Formula (6)),

g. (Φ)ₘ(−)ₙ(Φ)ₘ (Formula (7)),

h. (Φ)ₘ(+)ₙ(Φ)ₘ (Formula (8)),

i. (+)ₙ(Φ)ₘ(−)ₙ (Formula (9)), and

j. (−)ₙ(Φ)ₘ(+) (Formula (10)),
wherein:
(Φ) represents independently for each occurrence, a natural or non-natural amino acid comprising a hydrophobic side-chain; preferably alanine, valine, leucine, isoleucine or proline;
(+) represents independently for each occurrence a natural or non-natural amino acid comprising a side-chain that is cationic at physiological pH; preferably histidine, lysine or arginine;
(−) represents independently for each occurrence a natural or non-natural amino acid comprising a side-chain that is anionic at physiological pH; preferably aspartic acid or glutamic acid;
wherein the terminal amino acids are optionally substituted;
m for each occurrence represents an integer greater than or equal to 5; and
n for each occurrence represents an integer greater than or equal to 1; and further wherein the biologically active agent is encapsulated within the nanostructure.

The invention also encompasses a method of administering a biologically active molecule to a subject comprising administering to said subject a composition of the invention. In certain embodiments, the biologically active molecule has a controlled release profile.

The invention also includes methods for the preparation of the drug delivery composition.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1. Molecular modeling of amphiphilic, surfactant-like, self-assembling peptides. The peptide length is similar to biological phospholipids. The hydrophobic domain of the peptides consists of six alanines. Graphic illustrations were generated by VMD.

FIG. 2. HPLC chromatograms and MS spectra of the amphiphilic self-assembling peptides. MS/MS spectral analysis of the chromatographic peaks shows that the separated fragments of each peptide sample correspond to peptide purities of ˜92%.

FIG. 3 The AFM topography of amphiphilic, self-assembling peptide nanovesicles on mica. (Insets) size distribution histograms of peptide nanovesicles were generated from AFM image analysis.

FIG. 4. Inverse Laplace transform analysis of the experimental time correlation functions of the amphiphilic peptide nanovesicles in PBS pH 7.4 at 25° C. obtained by the CONTIN algorithm. Open circles: experimental data; solid lines: best fit curve obtained by inverse Laplace transform; solid squares: volume distributions of the hydrodynamic radii. Average magnitudes of the hydrodynamic diameters are shown for each distribution.

FIG. 5. Variation of the ζ-potential of the self-assembling peptide vesicles with electrolyte concentration. Values are the average of three measurements carried out at the stationary level at pH 7.4.

FIG. 6. Release kinetics of encapsulated carboxyfluorescein, CF, through peptide nanovesicles upon incubation in PBS pH 7.4, at 20° C.

FIG. 7 (Top panel). Nile Red emission spectra upon interaction with amphiphilic peptide nanovesicles in PBS pH 7.4, at 20° C. (Bottom panel) Release kinetics of encapsulated Nile red through peptide nanovesicles upon incubation in PBS pH 7.4, at 20° C.

FIG. 8. Effect of self-assembling amphiphilic peptides on Caco-2 cell viability after 3, 24, and 48 h in culture. Quantitative analyses represent means (n=5) with error bars showing the standard deviation. The percentage of viable cells between the four samples measured at day 2 are significantly different (P<0.01).

FIG. 9. Effect of lipid-like peptides on the permeation of FITC-dextran from the apical to the basolateral side of Caco-2 cell monolayers at (A) 0.2 mg/mL and (B) 1.0 mg/mL of peptides. Transepithelial resistance of Caco-2 cell intestinal epithelia in the presence of (C) 0.2 mg/mL and (D) 1.0 mg/mL of lipid-like peptides. Data are shown as mean±S.D. (n=4, <0.05 compared with HTC-dextran solution). The arrow indicates the time point at which the peptides were removed from the growth medium and shows the monolayer recovery process.

FIG. 10. Total transport of Rhodamine-123 in the apical side of the Transwell (secretory transport) after 180 minutes (n=3) in the presence of self-assembling peptides (A) below the CMC (0.02 mg/mL) and (B) above the CMC (0.2 mg/mL),

FIG. 11. Caco-2 cell culture treatment for 24 hr. Magnification 400×.

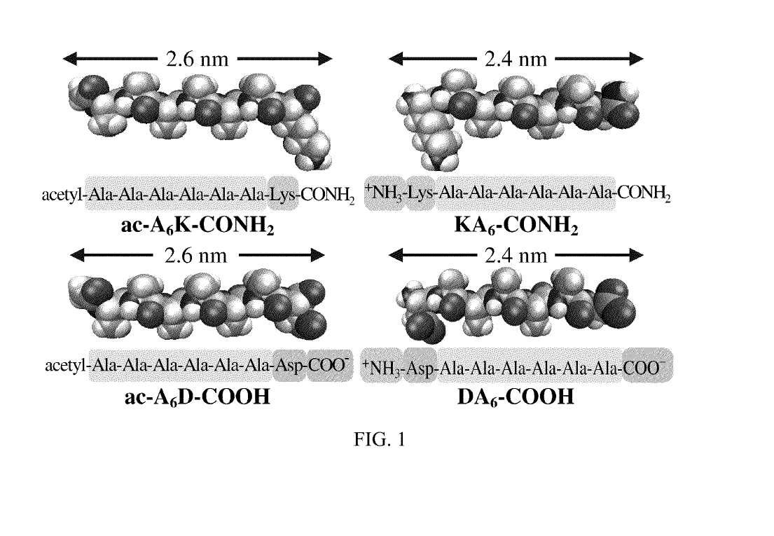

### DETAILED DESCRIPTION OF THE INVENTION

A description of preferred embodiments of the invention follows.

As used herein, “a” or “an” are taken to mean one or more unless otherwise specified.

The present invention is based on the discovery that self-assembled nanovesicles comprised of specific surfactant peptides can be used to encapsulate and release biologically active molecules, including hydrophilic and hydrophobic biologically active molecules.

Amphiphilic, surfactant-like, self-assembling peptides are functional materials, which, depending on the conditions, form a variety of nanostructures including nanovesicles, nanotubes, and nanovalves. Amphiphilic peptides, which have a hydrophilic head composed of aspartic acid or lysine and a six alanine-residue hydrophobic domain, were designed to have a length similar to that of biological lipids. A class of surfactant-like, self-assembling peptides, i.e., ac-A₆K-CONH₂ (SEQ ID NO: 1), KA₆-CONH₂ (SEQ ID NO: 2), and ac-A₆D-COOH (SEQ ID NO: 3) was designed to mimic lipid molecules of biological membranes and possess a hydrophilic head group, a hydrophobic tail, and length of 2-3 nm. Another peptide, the DA₆-COOH (SEQ ID NO: 4), was designed to have a charged head and tail separated by a hydrophobic domain of six alanines. These peptides, when dissolved in an electrolyte solution, self-assemble to minimize the interaction between the hydrophobic domains of the peptide and the polar environment. At physiological conditions, the ac-A₆K-CONH₂ (SEQ ID NO: 1), KA₆-CONH₂ (SEQ ID NO: 2), ac-A₆D-COOH (SEQ ID NO: 3) and DA₆-COOH (SEQ ID NO: 4) amphiphilic peptides self-assemble and form nanovesicles. In the case of self association of the negatively charged Ac-A₆D-COOH (SEQ ID NO: 1) and DA₆-COOH (SEQ ID NO: 4) peptides, AFM microscopic examination revealed necklace-like peptide vesicles supra-assemblies. The structures and the size of the vesicles depend on the amino acid sequence, the head type, and the charge distribution on the sequence. Release studies, described in more detail below, show that the peptide nanovesicle formulations can be used for the encapsulation and release of pharmaceutically active hydrophilic and hydrophobic compounds for drug delivery applications. As described below, the peptide nanovesicle systems are biocompatible and enhance drug delivery via a mechanism that involves interaction with the cell membrane P-glycoprotein system that controls the epithelial tight junctions.

Short amphiphilic self-assembling peptides are amenable to molecular design enabling modifications in the number, type, and order of amino acids on the peptide chain as well as incorporation of active peptide sequences to facilitate cell penetration and reactive chemical groups such as fluorescent dyes or biotin. The ease of production and the wide scope of modification allow for the synthesis of tailor-made sequences with tunable properties. When these amphiphilic peptide monomers undergo molecular self-assembly, the ultra-structural components formed (e.g., nanotubes or vesicles) can be further modified and tailored to confer functionality and in drug delivery applications in that peptide vesicles encapsulate therapeutic compounds and slowly release the load.

As used herein, the term “amino acid” encompasses a naturally or non-naturally occurring amino acid. Amino acids are represented by their well-known single-letter designations: A for alanine, C for cysteine, D for aspartic acid, E for glutamic acid, F for phenylalanine, G for glycine, H for histidine, I for isoleucine, K for lysine, L for leucine, M for methionine, N for asparagine, P for proline, Q for glutamine, R for arginine, S for serine, T for threonine, V for valine, W for tryptophan and Y for tyrosine.

The term “physiologic pH” is a pH of about 7. In some aspects, a physiological pH is a pH from about 6.6 to about 7.8. In yet other examples, a physiological pH is a pH from about 6.8 to about 7.6. In yet other examples, a physiological pH is a pH of about 7.0 to about 7.4.

The term “self-assembly” is a process of atoms, molecules or peptides forming regular shaped structures or aggregates in response to conditions in the environment, such as when added to an aqueous medium and/or when added to an aqueous medium at a physiological pH.

The term “critical aggregation concentration” or “critical micelle concentration” is the concentration above which the surfactant peptides aggregate or form regular shaped structures, such as micelles, nanotubes or nanovesicles.

A surfactant peptide is a short peptide with a hydrophilic head group and a lipophilic tail group. Surfactant peptides have been described, for example, in U.S. Pat. No. 7,179,784, U.S. Pat. No. 7,671,258, U.S. Patent Application Publication 20030176335 A1 and U.S. Patent Application Publication No. 20060211615 A1, the teachings of which are incorporated by reference herein. The class of peptides described in these patent publications were designed and shown to have the ability to spontaneously self-assemble to form stable nanostuctures. These short peptides (7 to 8 amino acids) have a structure similar to those observed in surfactant molecules with a defined hydrophilic head group constituted of charged amino acids and a lipophilic tail made out of hydrophobic amino acids such as alanine, valine, isoleucine or leucine. As a result, when dispersed in an aqueous solution, the amphiphilic peptides tend to self-assemble in order to isolate the hydrophobic tail from contact with water. The common feature for this self-assembly is the formation of a polar interface, which separates the hydrocarbon and water regions. In some embodiments, the surfactant peptide has a sequence of less than or equal to 10 amino acids. The hydrophilic head group is comprised of a polar and/or charged (either positively or negatively charged at physiological pH) amino acid. The hydrophobic tail group is comprised of a hydrophobic amino acid such as a non-polar and/or uncharged amino acid. In one embodiment, the hydrophilic amino acid is positively charged at physiological pH. In another embodiment, the hydrophilic amino acid is negatively charged at physiological pH. When dissolved in water, an aqueous solution, or in an ionic solution, the peptide surfactants undergo self-assembly to form a nanostructure such as micelles, nanovesicles or nanotubes. In some embodiments, the peptides of the inventive composition form a nanovesicle.

In certain aspects, the surfactant peptides used in accordance with the present invention are peptides having a formula selected from the group consisting of:

a. (Φ)ₘ(+)ₙ (Formula (1)),

b. (+)ₙ(Φ)ₘ (Formula (2)),

c. (Φ)ₘ(−)ₙ (Formula (3)),

d. (−)ₙ(Φ)ₘ (Formula (4)),

e. (−)ₙ(Φ)ₘ(−)ₙ (Formula (5)),

f. (+)ₙ(Φ)ₘ(+)ₙ (Formula (6)),

g. (Φ)ₘ(−)ₙ(Φ)ₘ (Formula (7)),

h. (Φ)ₘ(+)ₙ(Φ)ₘ (Formula (8)),

i. (+)ₙ(Φ)ₘ(−)ₙ (Formula (9)), and

j. (−)ₙ(Φ)ₘ(+) (Formula (10)),

wherein:

(Φ) represents independently for each occurrence, a natural or non-natural amino acid comprising a hydrophobic side-chain; preferably alanine, valine, leucine, isoleucine or proline;

(+) represents independently for each occurrence a natural or non-natural amino acid comprising a side-chain that is cationic at physiological pH; preferably histidine, lysine or arginine;

(−) represents independently for each occurrence a natural or non-natural amino acid comprising a side-chain that is anionic at physiological pH; preferably aspartic acid or glutamic acid;

wherein the terminal amino acids are optionally substituted;

m for each occurrence represents an integer greater than or equal to 5; and

n for each occurrence represents an integer greater than or equal to 1; under conditions suitable for self-assembly of the peptides into a nanostructure and allowing the nanostructure to be formed. Reading each of the Formulae (1) to (10) from left to right corresponds to the amino acid sequence from the N-terminus to the C-terminus.

In a further embodiment, the N-terminus of the surfactant peptide is blocked, e.g., acylated or acetylated. In an additional embodiment, the C-terminus of the surfactant peptide is blocked, e.g., esterified or amidated. In one aspect, the peptides having the formula 1, 3, 4, 5, 7, 8, or 10 at the N-terminal amino acid can be substituted by an acyl (e.g. acetyl or butyloxycarbonyl group) or other blocking group to remove the terminal charge. In another aspect, the peptides having the formula 1, 2, 4, 6, 7, 8, or 10 at the C-terminal amino acid can be substituted by an amino or alcohol group to form an amide or ester, or other blocking group to remove the terminal charge. One or both termini and any side chain residues can be optionally blocked or further substituted to modify (remove or add) charge, and/or increase or decrease hydrophobicity and/or hydrophilicity of the surfactant. Blocking groups that can be used to control charge, hydrophobicity or the ability to self-assemble in the surfactant include esters and amides of carboxylic acids, silyl ethers of alcohols, and acetals and ketals of aldehydes and ketones, respectively. The field of protecting group chemistry has been reviewed (Greene, T. W.; Wuts, P.G.M. Protective Groups in Organic Synthesis, 2nd ed.; Wiley: N.Y., 1991), which is incorporated by reference.

In certain embodiments, the surfactant peptide has the Formula (1) or the Formula (3). In additional aspects, the surfactant peptide has the Formula (1) or (3), wherein (Φ) is selected from the group consisting of alanine, valine, leucine, isoleucine and proline; in certain embodiments, (Φ) is alanine. In yet additional embodiments, the peptide has the Formula (1), wherein (+) is selected from the group consisting of histidine, lysine or arginine; in yet additional embodiments, (+) is lysine. In yet another embodiment, the peptide has the Formula (3), wherein (−) is selected from the group consisting of aspartic acid or glutamic acid; in yet additional embodiments, (−) is aspartic acid. In some aspects, the carboxylic acid of the N-terminal amino acid of the surfactant peptide is substituted with an acetyl group. In certain additional embodiments, the amino group of the C-terminal amino acid of the surfactant peptides is substituted with an amino group. In yet additional embodiments, the peptide has the Formula (1) or (3), wherein n is 1 and/or m is 5, 6 or 7. In yet additional aspects, the peptide has the Formula (1) or (3) wherein n is 1 and m is 6. In yet additional aspects, the peptide has the Formula (1) or (3) wherein n is 1 and m is 6, and (Φ) is alanine.

In some embodiments, the surfactant peptide is selected from the group consisting of ac-AAAAAAK-CONH₂ (SEQ ID NO: 1), KAAAAAA-CONH₂ (SEQ ID NO: 2), ac-AAAAAAD-COOH (SEQ ID NO: 3) and DAAAAAA-COOH (SEQ ID NO: 4). In yet additional embodiments, the surfactant peptide is ac-AAAAAAK-CONH₂ (SEQ ID NO: 1) or ac-AAAAAAD-COOH (SEQ ID NO: 3). The ac-A₆K-CONH₂ (SEQ ID NO: 1), KA₆-CONH₂ (SEQ ID NO: 2) and ac-A₆D-COOH (SEQ ID NO: 3) peptides have a hydrophobic tail of six alanine residues and a hydrophilic head which is an amino acid with a charged side group. A self-assembling amphiphilic peptide with polar groups at both the head and the tail is DA₆-COOH (SEQ ID NO: 4). Other exemplary surfactant peptides have been described, for example, in U.S. Pat. No. 7,179,784 and U.S. Pat. Application Publication No. 2009069547, the contents of each of which are hereby incorporated by reference.

The surfactant peptides described herein sequester in aqueous solutions and self-assemble to form nanostructures in a manner similar to that of lipid-based systems. For example, as shown below, addition of the surfactant peptides to phosphate buffered saline (PBS) solution containing 150 mM of electrolyte results in the formation of turbid suspension due to self-assembly of the peptide monomers. The peptide monomers self-assemble into nanostructures, for example, nanovesicles. The nanovesicles include a bilayer that is composed of the peptides' hydrophobic tails that self-assemble to form the vesicle. The biologically active molecule is encapsulated or in other words, entrapped or retained, within the nanostructure. The biologically active molecule is encapsulated within the nanostructure so long as it is retained within the structure for some period of time. As described in more detail below, the biologically active molecule can be released from the nanostructure after administration to the subject. The release kinetics depend on a number of factors which are discussed in more detail below.

In certain embodiments, the self-assembled nanostructure is a nanovesicle. As described below, the diameter of the nanovesicles can be determined using Atomic Force Microscopy (AFM) and/or Dynamic Light Scattering (DLS), for example. In some examples, the nanovesicle has an average diameter of about 30 to about 800 nm, about 60 nm to about 500 nm, or about 80 nm to about 300 nm. In yet other examples, the nanovesicle has an average diameter of about 80 nm to about 300 nm, as measured by Dynamic Light Scattering (DLS). In certain other examples, the nanovesicle has an average diameter of about 90 nm to about 200 nm, as measured by DLS.

In yet additional aspects, the composition comprises a nanostructure wherein the nanostructure comprises a plurality of surfactant peptides, wherein the peptide monomers are selected from the group consisting of ac-AAAAAAK-CONH₂ (SEQ ID NO: 1), KAAAAAA (KA₆-CONH2), ac-AAAAAAD-COOH (SEQ ID NO: 3) and DAAAAAA-COOH (SEQ ID NO: 4), and wherein the nanostructure is a nanovesicle. Table 2 shows the vesicle diameter for nanovesicles comprised of peptides having SEQ ID NOs: 1 to 4 as measured suing AFM or DLS. The nanovesicles can, for example, have an average diameter within the range described below in Table 2. In certain embodiments, the peptides have the amino acid sequence of ac-AAAAAAK-CONH₂ (SEQ ID NO: 1) and the nanovesicle has an average diameter of about 100 nm to 135 nm; in some embodiments, the average diameter is as measured by DLS. In yet additional embodiments, the peptides having the amino acid sequence of ac-AAAAAAK-COOH (SEQ ID NO: 3) and the nanovesicle has an average diameter of about 80 nm to about 120 nm; in some embodiments, the average diameter is as measured by DLS.

In yet additional embodiments, the biologically active molecule is hydrophobic. In further embodiments, the biologically active molecule is hydrophilic.

The compositions of the present invention can further comprise an additional molecule including, but not limited to, a liposome. The peptides can also be modified to incorporate one or more other molecules including, but not limited to a sugar and/or a biologically active motif, such a cell signaling and/or cell penetrating amino acid.

As discussed above, the invention also encompasses a method of administering a biologically active molecule to a subject comprising administering to said subject a composition of the invention. In some embodiments, the biologically active molecule is delivered to the gastrointestinal tract or across the blood brain barrier of said subject. In certain aspects, the biologically active molecule is delivered to the gastrointestinal tract and the surfactant peptides are negatively charged. In some embodiments, the biologically active molecule is delivered across an epithelial monolayer. In yet additional embodiments, the biologically active molecule is delivered across an epithelial monolayer via an interaction of the drug delivery composition and/or the nanostructure with P-glycoprotein.

The nanostructures described herein can be used to administer a biologically active agent to a subject. The term “subject” includes animals and humans. In some embodiments, the nanostructures are used to administer a biologically active agent to a human subject.

The term “controlled release profile” as used herein refers to the characteristics of the release of the biologically active molecule from the composition described herein, and will be designed according to the specific application of the formulation obtained. Controlled release encompasses delayed, sustained or prolonged release, and the like. Use of the nanostructures described herein allows a controlled release of the biologically active molecule after administration to the subject. The selection of the desired release profile depends on considerations known to those skilled in the art, such as the disease or indication to be treated, the treatment regimen, the patient to be treated, the route of administration and/or the site of administration, etc. As described in more detail below, the release kinetics of the biologically active molecule can be controlled, for example, by varying the size of the nanostructures (e.g., nanovesicles), their loading capacity, and/or by altering the charge of the surfactant peptide monomers that self-assemble to form the nanostructures. The controlled release formulation described herein can be an injectable preparation, an implant, an oral preparation (e.g., powders, granules, capsules, tablets, syrups, emulsions, suspensions), and/or a topical formulation.

Exemplary biologically active molecules include, for example, small molecules, peptides, and nucleic acids. It will be understood that the term “peptides” encompasses proteins (including, for example, cytokines, hormones and clotting factors) and antibodies. Nucleic acids that can be administered according to the present invention include, but are not limited to, those comprising: recombinant nucleic acids; genomic DNA, cDNA, and RNA.

Formulations of the present invention include those suitable for oral, nasal, topical (including buccal and sublingual), rectal, vaginal and/or parenteral administration. The formulations can conveniently be presented in unit dosage form and may be prepared by any methods well known in the art of pharmacy. The amount of active ingredient or biologically active molecule produce a single dosage form will vary depending upon the host being treated, the particular mode of administration.

Methods of preparing these formulations or compositions can include the step of bringing into association a biologically active molecule with the surfactant peptides described herein in an aqueous solution under conditions suitable for self-assembly, and, optionally, can include one or more accessory ingredients. Conditions which permit self-assembly have been described in the literature and are described in detail in the Examples below. Such conditions have been described, for example, in U.S. Pat. No. 7,179,784, U.S. Pat. No. 7,671,258, U.S. Patent Application Publication 20030176335 A1 and U.S. Patent Application Publication No. 20060211615 A1, the teachings of which are incorporated by reference herein. Such conditions include, for example, a suitable pH for self-assembly (for example, at or near physiological pH) and/or a suitable concentration of the self-assembling peptides (for example, at or above the CMC).

The invention is illustrated by the following examples which are not meant to be limiting in any way.

### EXEMPLIFICATION

### Materials and Methods

### Surfactant-Like, Self Assembling Peptides.

The amphiphilic peptides acetyl-AAAAAAK-CONH₂ (ac-A₆K-CONH₂; SEQ ID NO: 1), KAAAAAA-CONH₂ (KA₆-CONH₂; SEQ ID NO: 2), acetyl-AAAAAAD-COOH (ac-A₆D-COOH; SEQ ID NO: 3), and DAAAAAA-COON (DA₆-COOH; SEQ ID NO: 4) were purchased (SynBioSci, Livermore, Calif.). The purity of the peptides was about 92% as determined by electrospray ionization-quadrupole-time-of-flight (ESI-Q-TOF) mass spectrometry. Peptides were received in powder, dispersed in phosphate buffer saline, (PBS, 100 mM KH₂PO₄, 10 mM Na₂HPO₄, 137 mM NaCl, 2.7 mM KCl at pH 7.4) and sonicated for 10 min using a bath sonicator to facilitate solubilization and dispersion. The peptide solutions were then allowed to equilibrate for 15 min to allow for self association of the monomers, filtered through Amicon Ultra 3 kDa MWCO to remove non-associated peptide monomers and other synthesis residual molecules, resuspended, filtered through a 0.4 urn filter to remove large peptide vesicle aggregates, and stored at room temperature for further studies.

### Mass Spectrometry

The peptides in solution were characterized by electrospray ionization-quadrupole-time-of-flight (ESI-Q-TOF) mass spectrometry. Separation of the peptide fragments was performed using a Zorbax 300 Extend-C18 column (Agilent Technologies, Palo Alto, Calif.) and an Agilent 1200 series chromatography system coupled to Agilent 6510 Q-TOF (spray voltage of 3.8 kV, gas temperature of 275° C., nebulizer gas at 10 psi, and a drying gas of 4 L/min). Positive ion data-dependent acquisition range in the scan mode was 100-1799 mlz and for the MS/MS mode 100-2000 mi. The charge priority of MS/MS acquisition was +2 (charges), +3, >+3, unknown, +1. Data were collected and analyzed using the Agilent MassHunter software (Agilent, Technologies, Palo Alto, Calif.) Peptide identification, assignment, and possible amino acid chemical modifications were performed by inspection of the MS/MS spectra and by using the ProteinProspector software (http:prospector.ucsfedu).

### Critical Micelle Concentration (CMC) of the Amphiphilic Peptides.

The CMC of the amphiphilic peptides was determined by Dynamic Light Scattering (DLS) experiments (PDDLS/Batch setup, Precision Detectors, Franklin, Mass.). Solutions of different peptide concentrations were prepared in PBS and were filtered through 0.4 urn pore size filters prior to measuring. Scattered light was detected at 90° and the number of photons reaching the avalanche photodiode was recorded. The solvent viscosity and the refractive index of the buffer were taken as 0.894 cP and 1.33, respectively, at 20° C. Data were acquired in triplicates and processed by the Precision Deconvolve program,

### Particle Size Determination.

Prior to measuring particle sizes by DLS, the peptide vesicle formulations in PBS were filtered through 0.4 um filter to remove vesicle aggregates. Normalized intensity-time correlation functions g⁽²⁾ (q,t)=I(q,t)I(q,0))/I(q,01))² were measured over a broad time scale (from 10⁻⁷ s to 10⁴ s) using a full multiple tau digital correlator (ALV-5000FAST) with 280 channels spaced quasi-logarithmically. The scattering wavevector q=4πn sin(θ/2)/λₒ depends on the scattering angle θ (herein we used 0=90°), the laser wavelength λₒ, and the refractive index of the medium n, We used the 671 nm line from a diode pumped solid state laser which operated at a power of less than 5 mW. The scattered light was collected by a single-mode optical fiber (coherence factor ˜0.95), transferred to an avalanche photo-detector and then to the digital correlator for analysis. Accumulation times were of the order of few seconds due to the strong scattered intensity from the dispersions. To check the reproducibility of the results, several time correlation functions were recorded for each peptide vesicle system dilute suspensions the normalized electric-field time auto-correlation function g⁽¹⁾=(E(q,t)E*(q,0))/(E(q,0))² is related to the experimentally recorded intensity autocorrelation function g⁽²⁾ (q,t)² through the Siegert relation [13] g⁽²⁾ (q, t)=B[1+f*|g(1)(q,t)|² where B describes the long delay time behavior of g⁽²⁾ (q, t) and f* represents an instrumental factor obtained experimentally from measurements of a dilute polystyrene/toluene solution. In our case, the optical fiber collection results in f*˜0.95. The electric-field time correlation function g⁽¹⁾(t) (for simplicity we drop the q-dependence) was analyzed as a weighted sum of independent exponential contributions, i.e., g⁽¹⁾∫L(τ)exp(−t/τ)dτ=∫L(ln τ)exp(−t/τ)d in τ. The distribution of relaxation times L(lnr)=τL(τ) was obtained by inverse Laplace transformation of g<′>(q,t) using the CONTIN algorithm [14].

The apparent hydrodynamic radii of the suspended particles were determined using the Stokes-Einstein relation Rₕ=k_{B}T/6πηD, where k_{B} is the Boltzmann constant, η is the viscosity of the solvent, and D the diffusion coefficient of the particle. The latter is determined by D=1/t q². where τ is the relaxation time of g(¹⁾ (q,t).

### Microelectrophoretic Mobility Measurements.

To determine the ζ-potential of the peptide nanovesicle formulations we used a Nano ZetaSizer (Malvern Instrument, UK) equipped with an avalanche photodiode as detector and a 4 mW He—Ne laser operated at 633 nm. Data were acquired by laser Doppler velocimetry and phase analysis light scattering (PALS). The self-assembling peptides were dispersed in PBS at a concentration of 20 mg/mL, which is above their CMC, and equilibrated for 30 min at 25° C. prior to detection. Microelectrophoretic data were obtained using the Henry-Smoluchowski equation.

### Atomic Force Microscopy (AFM).

For the AFM experiments, 3 uL were removed from the peptide vesicle dispersions (20 mg/mL of peptides in PBS that has been filtered through 0.2 um filters) and deposited on a freshly cleaved mica surface (G250-2 mica sheets 2.5×2.5×0.015 cm; Agar Scientific Ltd, Essex, UK). The bare surface of mica is smooth (rms is about 0.4 nm). Before imaging, each sample was left on mica for 1 min, rinsed with 200 uL of water (Millipore), and dried in a nitrogen gas stream. Images were obtained immediately in air using a Veeco MultiMode NanoScope 3D Controller Scanning Probe Microscope (Digital Instruments, Santa Barbara, Calif.) operated in tapping mode. AFM imaging (512×512 pixels) was performed using soft silicon probes (FESP; nominal length lₙₒₘ=225 um, width wₙₒₘ=28 um, tip radius Rₙₒₘ=8 nm, resonant frequency νₙₒₘ=75 kHz, spring constant kₙₒₘ=2.8 N m⁻¹; Veeco Instruments SAS, Dourdan, France). Typical scanning parameters were tapping frequency 73 kHz, integral and proportional gains 0.2 and 0.4, respectively, set point 0.7-1.0 V, and scanning speed 1.0-1.5 Hz. The Nanoscope image processing software was used to obtain height patterns, cross sections, and MIS of the peptide nanovesicles. Images were collected from two different samples at random spot surface sampling (at least five areas).

### Release Through Peptide Vesicles.

To determine whether the peptide vesicles can be used for drug delivery we measured the release kinetics of the hydrophilic fluorescent probe 5,6-carboxyfluorescein (CF) through the peptide bilayer assemblies. The peptide powder was added into the CF solution in PBS and the suspension was probe-sonicated for 10 min followed by equilibration for 1 hour. Encapsulated CF is strongly quenched and therefore, only the released CF contributes to the fluorescence intensity signal. Released CF was removed by centrifugation at 11,000 rpm for 25 min using Microcon YM-10 membrane tubes (10 kDa cut-off). Then the vesicles were re-suspended in PBS and incubated for 1 hour at 20° C. The newly released CF during incubation was collected by centrifugation through the Microcon membrane and the fluorescence intensity was measured. This process was repeated every 1 hour and a graph was created to show the release kinetics of CF as a function of time. All measurements were carried out in a Perkin-Elmer LS-50B spectrophotometer at 20° C. using quartz cuvettes of 1 cm (path length. The excitation wavelength was at 470 nm and the emission maximum was observed at 520 nm. The excitation and emission slit widths were set at 5.0 and 2.5 nm, respectively.

### Hydrophobic Drug Uptake.

The hydrophobic fluorescent probe Nile red was used as a probe molecule to study the peptide nanovesicle bilayer. Nile red shows strong solvate-chromism and the emission spectrum varies depending on its local microenvironment. The “thin film” method was used for the preparation of the dispersions. Briefly, 0.2 mg of the amphiphilic peptide was dissolved in ethanol and subsequently the organic solvent was removed under vacuum in a rotary evaporator. Then the produced thin film was rehydrated with 1 mL of PBS, pH 7.4 containing 3.14 uM Nile red. Prior to analysis the dispersions were bath sonicated for 30 minutes. All spectra were recorded at room temperature with excitation and emission slit widths set at 5 nm (RF-5301 PC Shimatzu spectrophotometer). The excitation wavelength was at 546 nm and the emission spectra were recorded from 550 to 700 nm.

### Cell Cultures.

Caco-2 cells (passage 40) were grown at 37° C. in tissue culture flasks with Dubelco's Modified Essential Medium (DMEM) supplemented with 10% v/v FBS (Fetal Bovine Serum) 1% nonessential amino acids and 100 ug/mL penicillin and streptomycin in humidified atmosphere containing 5% v/v CO₂. The medium was changed every 2-3 days until cells reach 80% confluence and then were subcultured by trypsinization.

### Cell Viability MTT Assay.

The effect of self-assembling peptides on cell viability and cell proliferation was investigated using the MTT colorimetric assay (Sigma). Caco-2 cells were cultured into the wells of 96-well plates using growth medium until a density of 4×10³ cells/cm² was reached. Then we replaced the growth medium with fresh growth medium containing 0.2 or 1.0 mg/mL self-assembling peptides and the cells were incubated for 3, 24 and 48 h at 37° C. Then 10 uL of MTT reagent was added in each of the wells and the plates were incubated for 3 hat 37° C. Then 100 uL of the detergent solution was added and the plates were stored in the dark for 3 h to dissolve the formazan product. The optical density was measured at 600 nm in a Teknika® ELISA Plate spectrophotometer.

### Transepithelial Resistance and Cell Monolayer Permeability.

Caco-2 cells were seeded at a density of 2×10⁵ cells/cm² on 24-well 6.5 mm Transwell polyester membrane inserts (0.4 um pore size, Corning Costar, Cambridge, Mass.). The medium was changed every 2-3 days in both sides of the Transwell membrane. After 20 days the growth medium was replaced with 10 mM HBSS/HEPES buffer pH 7.4 and studies of the cell-monolayer integrity and permeability were performed.

Transepithelial electrical resistance (TEER) of the Caco-2 monolayers in 10 mM HBSS/HEPES buffer pH 7.4 was monitored every 30 min for 5 h using a Millicell-ERS® device (Millipore) in the presence of 0.2 and 1.0 mg/mL, self-assembling peptides added to the cell growth medium. At 180 min, the 10 mM HBSS/HEPES buffer pH 7.4 medium was replaced with FBS-free DMEM and the transepithelial resistance was measured for another 120 min to assess the ability of cells to recover from treatment. All measurements were done in triplicates.

For the permeability assay, we added in the apical side of the Transwell 1 mg/mL FITC-dextran MW 4.4 kDa (Sigma) in 10 mM HBSS/HEPES buffer pH 7.4 with and without 0.2 or 1.0 mg/mL of self-assembling peptides. At each time point, samples were withdrawn from the basorateral side and the amount of FITC-dextran was measured using a fluorescence 96-well plate reader. The excitation and emission wavelengths were 490 nm and 530 nm, respectively. Results were expressed as cumulative transepithelial transport of FITC-dextran as a function of time. All measurements were done in triplicates and expressed as mean±S.D. The apparent permeability coefficient was calculated according to Pₐₚₚ=(dQ/dt)/(dt)/(ACₒ) in cm/s, where dQ, is the cumulative amount of FITC-dextran in the basolateral side at time t, (dQ/dt) in pmol/s is the permeability rate, Cₒ is the initial concentration of FITC-dextran in the apical side and A is the area of the monolayer. The enhancement ratio R equals to R=(P_{app(peptide)}/P_{app(control)}).

Analysis of variance (ANOVA) was performed to determine whether the differences observed in the calculated apparent permeability coefficients were statistically significant. Significant F values are indicative of a significant difference in the calculated apparent permeability coefficients when the probability is less than 0.05 (P<0.05).

### Transepithelial Transport of Rbodamine-123.

Transepithelial transport through Caco-2 monolayers was monitored using 0.5 uM Rhodamine-123 added to the basolateral side of the Transwell. Self-assembling peptides were added in the basolateral side of the Transwell at concentrations above and below the peptides' CMC values at 0.016 and 0.16 mg/mL. Control experiments were performed with the addition of 1% SDS or 100 uM Verapamil which is a P-glycoprotein inhibitor, Every 30 mM samples were withdrawn from the apical side and analyzed by HPLC (Shimadzu® HPLC equipped with Waters Xterra. C18 150 mm×46 mm, 3.5 um analytical column, flow rate 0.85 ml/min, detector was set at 500 nm) [15]. The mobile phase, which consisted of acetonitrile, 20 mM sodium acetate buffer pH 4.0, and water containing 1.5 mM. TBA (502030 v/v/v), was filtered through 0.45 um filter and degassed by ultrasonication prior to use. Before the end of the experiment, the transepithelial resistance was measured to ensure that the cell monolayer integrity had not been affected by the experimental conditions.

### Immunocytochemistry.

Caco-2 cells (passage 40) were grown on cover slips to reach 70-80% confluence before interaction with 0.2 or 1.0 mg/mL of the self-assembling peptides for 24 hr. Untreated. Caco-2 cells served as controls. At the end of the incubation period the cover slips were rinsed with PBS and fixed with 4% w/v paraformaldehyde for 30 min. at room temperature. The cells were then treated with 0.3% Triton X-100 in PBS for 10 min followed by addition of blocking buffer (5% BSA, 10% NGS) and incubation for 30 min at room temperature. The cover slips were then rinsed with PBS and incubated with primary mouse monoclonal E-cadherin antibody (1:100) (Novocastra Laboratories Ltd, UK) for 1 h at room temperature, washed again with PBS, incubated with secondary Alexa-Auor 568 goat anti-mouse antibody (1:1000) (Invitrogen) for 1 h, and washed with PBS. DAPI was used to stain cell nuclei. Imaging was performed by Nikon D-Eclipse fluorescence microscope.

### Results and Discussion

The rationale for the design of the peptide sequences that we tested in this work was to resemble the length and the structure of the phospholipids that are present in biological membranes. The ac-A₆K-CONH₂ (SEQ ID NO: 1), KA₆;-CONH₂ (SEQ ID NO: 2), and ac-A₆D-COOH (SEQ ID NO: 3) peptides have a hydrophobic tail comprised of six alanines and a hydrophilic head which is an amino acid with charged side group. A self-assembling amphiphilic peptide with polar groups at both the head and the tail, DA₆-COOH (SEQ ID NO: 4), was also synthesized and the results of the morphological, physicochemical, and peptide-cell interaction analyses were compared with that of the surfactant-like peptides. Like lipids and anionic surfactants, lipid-like peptides sequester in aqueous solutions and self-assemble to form nanostructures in manner similar to that occurring in lipid-based systems [6,16]. Addition of surfactant-like peptides to PBS solution containing 150 mM of electrolyte results in the formation of a turbid suspension due to self-assembly of the peptide monomers.

### Mass Spectrometric Analysis of the Peptides

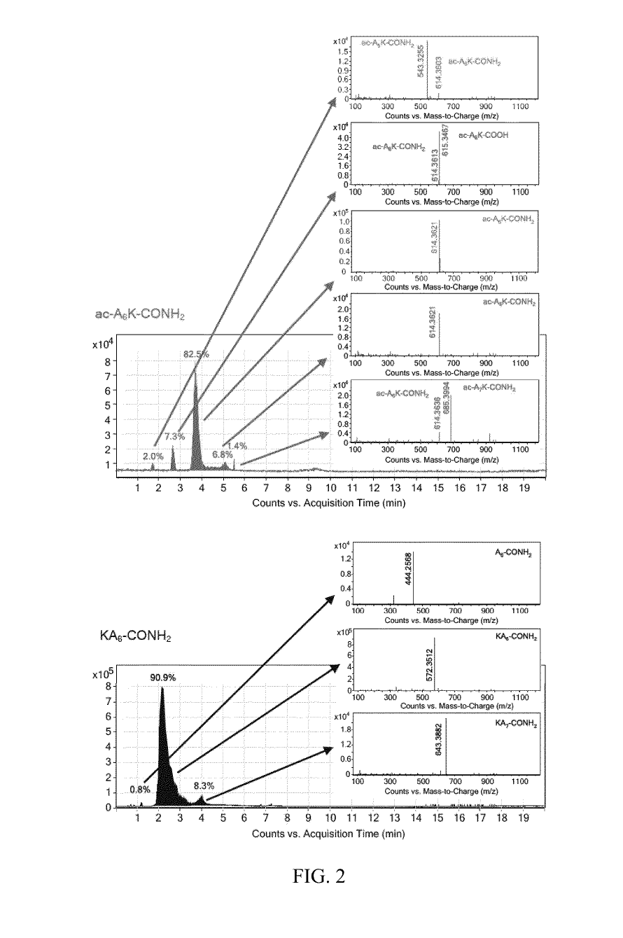

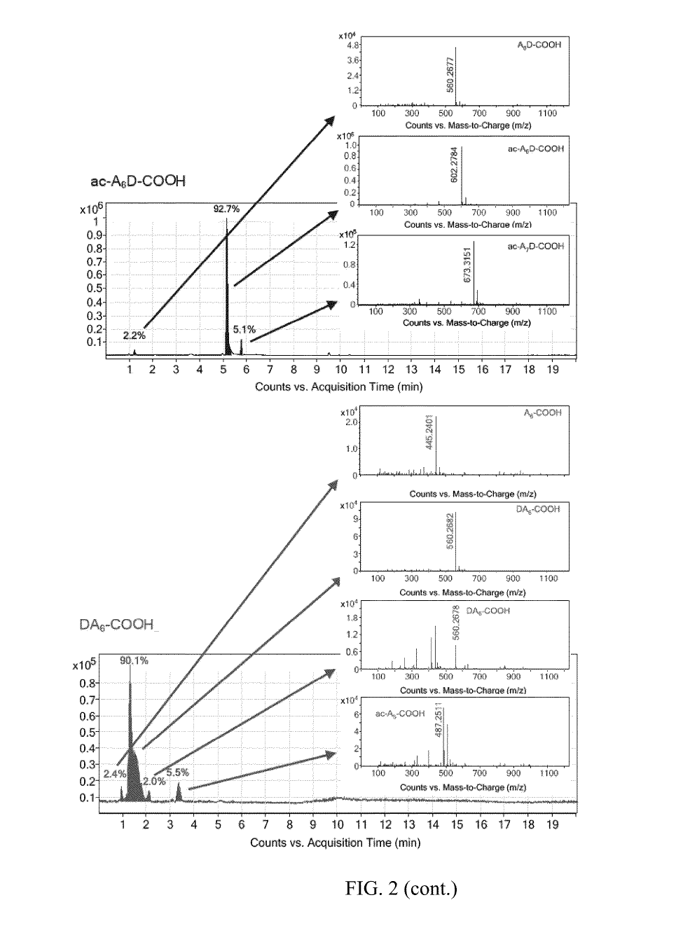

The peptides were separated by HPLC and analyzed by ESI-Q-TOF mass spectrometry (FIG. 2). Quantitative analysis using combined peak integration of the extracted chromatograms for all charged states reveals that the purity of the peptides was between 90% and 92%. As may be seen in the MS spectra in FIG. 2, the samples contain traces of shorter and, in some cases, of longer peptides which is common in synthetic peptide preparations. Non assigned peaks which are present in the MS spectra are due to intra-source fragmentation of the +1 charged peptides.

AFM imaging

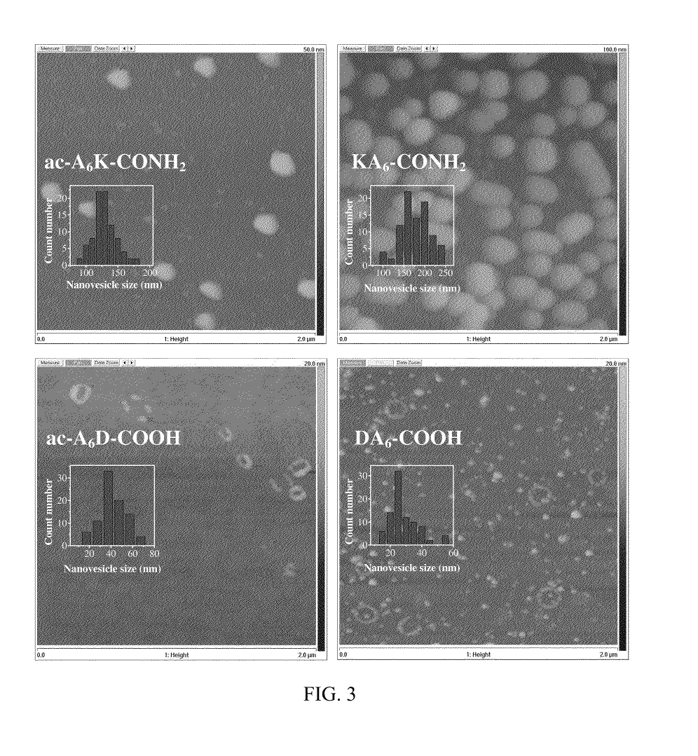

We used AFM to study the morphology of the amphiphilic peptide assemblies. Previously, we reported the formation of vesicles and nanotubes depending on the experimental conditions [6, 7, 10]. Herein, the experimental conditions consistently led to the formation of nanovesicles. FIG. 3 shows that the size of the amphiphilic peptide assemblies depended on the type of the peptide used. Size distribution analyses of the peptide vesicles showed that self-assembly of the lysine-containing, positively charged ac-A₆K-CONH₂ (SEQ ID NO: 1) and KA₆-CONH₂ (SEQ ID NO: 2) peptides results in larger particles compared to those obtained from association of negatively charged ac-A₆D-COOH (SEQ ID NO: 3) and DA₆-000.11 (SEQ ID NO: 4) peptides (FIG. 3, insets, and Table 1), This is likely due to the smaller side chain of aspartic acid compared to lysine which allows for better packing of the peptides in the nanovesicle bilayer.

In the case of the ac-A₆D-COOH (SEQ ID NO: 3) and DA₆-COOH (SEQ ID NO: 4) peptides, the image topology revealed necklace-like ultra-structures. Such formations are commonly observed for nanoparticle colloidal solutions when a drop of liquid dries on a solid surface and may be described by two dimensional crystallization laws [17] or the coffee-ring effect [18] at the nanometer scale due to evaporation of the solvent. The association of the smaller ac-A₆D-COOH (SEQ ID NO: 3) and DA₆-COOH (SEQ ID NO: 4) peptide nanovesicles towards the formation of such ultra-structures instead of the presence of individual peptide nanovesicles as in the case of ac-AK-CONH₂ (SEQ ID NO: 1) and KA₆-CONH₂ (SEQ ID NO: 2) may be driven by surface energy requirements of the system. The average diameter of the necklace-like formations consisted of ac-A₆D-COOH (SEQ ID NO: 3) and DA₆-COOH (SEQ ID NO: 4) peptide nanovesicles is 200±11 nm and 159±26 nm, respectively. Furthermore, image analysis showed that ac-A₆D-COOH (SEQ ID NO: 3) necklaces are composed of 23±3 of the smaller ac-A₆D-COOH (SEQ ID NO: 3) nanovesicles whereas those composed of the larger DA₆-COOH (SEQ ID NO:4) nanovesicles have 11±2 nanovesicles per necklace.

These observations prompted inquiry about the mechanism of necklace formation. Changing the drying conditions during AFM sample preparation revealed metastable nanovesicle clusters suggesting that prior to necklace formation ac-A₆D-COOH (SEQ ID NO: 3) and DA₆-COOH (SEQ ID NO: 4) peptide nanovesicles formed loosely bound clusters with diameter 101+11 and 135±24 nm, respectively. During the drying process of the sample, the negatively charged peptide nanovesicle aggregates disassembled and spread on the like charged mica surface leading to the formation of the nanovesicle necklaces. In the case of ac-A₆D-COON (SEQ ID NO: 3) nanovesicles, the formation of larger diameter necklaces is probably due to the smaller size and the higher number of individual peptide nanovesicles, i.e., 23, per ac-A₆D-COOH (SEQ ID NO: 3) nanovesicle aggregate compared to the DA₆-COOH aggregates which required 11 peptide nanovesicles of larger diameter. Theoretical calculations revealed that the size of the ac-A₆D-COOH (SEQ ID NO: 3) and DA₆-COON (SEQ ID NO: 4) nanovesicle aggregates, i.e., 101±11 and 135±24 nm, respectively correlates well with 23 and 11, respectively tightly packed individual nanovesicles with diameter 28±9 nm and 43±11 nm, respectively.

### Dynamic Light Scattering

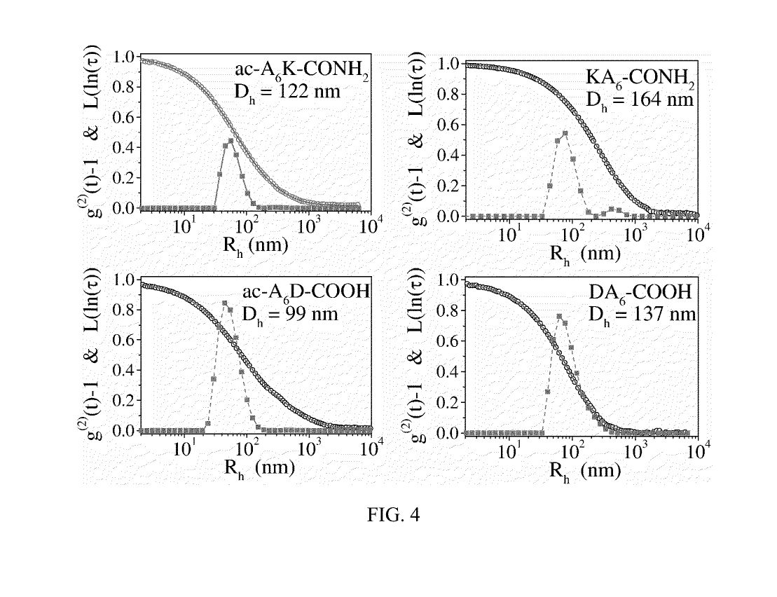

The hydrodynamic radius, Rₕ, of the peptide nanovesicles was determined by DLS. The intensity time correlation functions and the corresponding inverse Laplace transform analyses of dispersed amphiphilic peptide vesicles in PBS pH 7.4 at 25° C. is shown in FIG. 4. Solid lines represent the best fit results which were obtained using the CONTIN algorithm. Inverse Laplace transformation analysis of the light scattering data yielded monomodal peptide vesicle size distributions except in the case of KA₆-CONH₂ (SEQ ID NO: 2) vesicle suspensions in which peak analysis showed the presence of nanovesicles with average diameter of 164 nm and 906 nm; the latter probably represent aggregates of individual vesicles (FIG. 4). The average diameter of the ac-A₆K-CONH₂ (SEQ ID NO: 1) peptide nanovesicles is 122 nm (FIG. 4 and Table 2). DLS measurement of ac-A₆K-CONH₂(SEQ ID NO: 1) and KA₆-CONH₂ (SEQ ID NO: 2) peptide nanovesicles are in good agreement with the size determined by AFM of nanovesicles deposited on mica. However, DLS analysis of ac-A₆D-COOH (SEQ ID NO: 3) and DA₆-COOH (SEQ ID NO: 4) peptide vesicles revealed particles with diameter 97 nm and 137 nm, respectively. These values deviated from the vesicle diameters determined by AFM analysis for individual ac-A₆D-COOH (SEQ ID NO: 3) and DA₆-COOH (SEQ ID NO: 4) peptide nanovesicles which on AFM showed necklace-like formations and nanovesicle clusters. However, inspection of the data shows that the DLS data analysis resulted in particle diameters that are similar to those determined for the nanovesicle clusters suggesting that these clusters are stable in the PBS solution in which DLS was performed (Table 1, FIG. 4).

Particle size determination by DLS represents the hydrodynamic diameter of the peptide vesicles in solution whereas AFM imaging provides information about peptide vesicles in the dry state. We found that there is a good agreement between the calculated vesicle sizes from DLS and AFM suggesting that vesicle shrinking is negligible during the drying process that precedes AFM scanning. Because of tip broadening effects the actual diameter of the vesicles is smaller than that measured by AFM [19] and therefore, the vesicle diameter observed by AFM correlates well with the hydrodynamic diameter determined by DLS data analysis.

### Microelectrophoretic Mobility Measurements

Electrostatic phenomena are important in many biological processes. The introduction of charge on nanoparticulate systems can alter their biological and physicochemical properties [20]. The colloidal stability of vesicles dispersed in a polar solvent is associated with electrostatic repulsions between like charged particle surfaces. To determine the stability of the peptide nanovesicle formulations in PBS we measured their surface potential values.

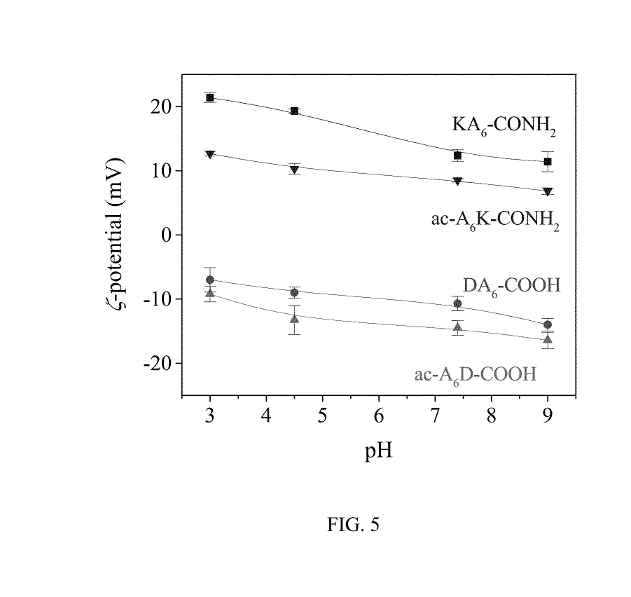

At pH 7.4, the positive ζ-potential values obtained for vesicles consisting of ac-A₆K-CONH₂ (SEQ ID NO: 1) (i.e., 8.5±0.3 mV) and KA₆-CONH₂ (SEQ ID NO: 2) (i.e., 12.4±0.9 mV) are due to the net positive charge of peptide monomers. The fact that we measured higher (t-test, p<0.05) ζ-potential values for the KA₆-CONH₂ (SEQ ID NO: 2) compared to ac-A₆K-CONH₂ (SEQ ID NO: 1) vesicles is consistent with the peptides' respective net charges i.e., (+2) for KA₆-CONH₂ (SEQ ID NO: 2) and (+1) for ac-A₆K-CONH₂ (SEQ ID NO: 1) in PBS pH 7.4. Nanovesicles composed of ac-A₆D-COOH (SEQ ID NO: 3) and DA₆-COOH (SEQ ID NO: 4) peptides exhibit negative ζ-potential values of −15.4±1.1 mV and −14.0±1.1 mV, respectively, consistent with the net charge of ac-A₆D-COOH (SEQ ID NO: 3), i.e., (−2) and DA₆-COOH (SEQ ID NO: 4), i.e., (−1) at pH 7.4. The electrophoretic behavior of the peptide nanovesicles in PBS as a function of pH at constant ionic strength is shown in FIG. 5. As the pH increases, the surface charge of the positively charged ac-A₆KCONH₂ (SEQ ID NO: 1) and KA₆-CONH₂ (SEQ ID NO: 2) peptide vesicles shifts to lower positive-potential values, or to more negative κ-potential values in the case of ac-A₆D-COOH (SEQ ID NO: 3) and DA₆-COOH (SEQ ID NO: 4) vesicles due to increased adsorption of hydroxyl groups on the particle surface in alkaline solutions.

### Release Through Peptide Vesicles for Drug Delivery Applications

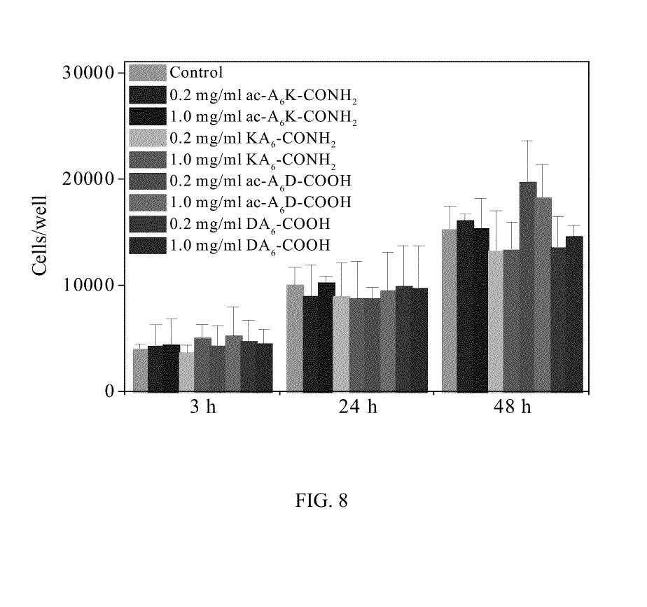

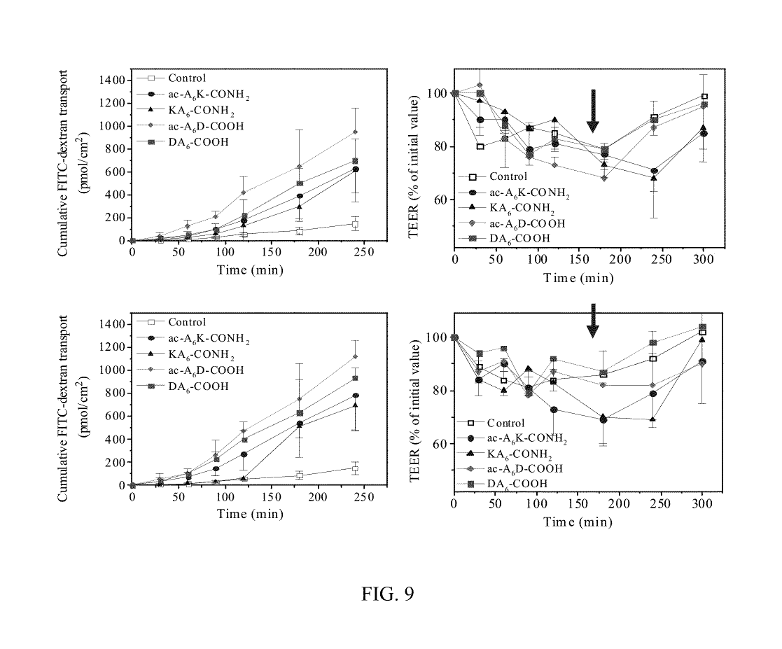

To determine whether the self-assembling peptide nanovesicles can be used in drug delivery applications we measured the release kinetics of the fluorescent probe CF through the nanovesicles bilayer. FIG. 8 shows that peptide vesicles consisting of the surfactant-like ac-A₆K-CONH₂ (SEQ ID NO: 1) peptide retained CF entrapped inside the vesicle for prolonged periods of time. At the conditions employed in the release experiments (i.e., centrifugation and resuspension of the vesicles) the CF release through the ac-A₆K-CONH₂ (SEQ ID NO: 1) peptide nanovesicles was approximately 50% after 7 h. In the case of ac-A₆D-COOH (SEQ ID NO: 3) based formulations, the vesicles retained 13% of the initially loaded CF in the respective period of time. Vesicles composed of KA₆-CONH₂ (SEQ ID NO: 2) and DA₆-COOH (SEQ ID NO: 4) peptides completely released CF within 2 to 3 hours.

We asked whether the difference between the release kinetics of encapsulated CF through ac-A₆K-CONH2 (SEQ ID NO: 1) and ac-A₆D-COOH (SEQ ID NO: 2) nanovesicles was solely due to electrostatic interactions between the negatively charged CF and the surface of the vesicles. Ac-A₆K-CONH₂ (SEQ ID NO: 1) is acetylated and amidated and the C- and N-termini, respectively and at pH 7.4 carries only positive charge at the ε-amine group of lysine. The ac-A₆D-COOH (SEQ ID NO: 3) peptide carries two negative charges at the C-terminus due to the negatively charged carboxyl groups of aspartic acid. In a separate experiment, we added CF to a dispersion containing pre-formed peptide nanovesicles. Following centrifugation, removal of the supernatant, and resuspension of the vesicles we observed minimal CF fluorescence in the system which suggests that the observed release profiles are due to encapsulation and slow release of CF through the ac-A₆K-CONH₂ (SEQ ID NO: 1), and to a lesser extent through ac-A₆D-COOH (SEQ ID NO: 3) peptide bilayers. These results suggest that peptide vesicles can be used for drug or gene delivery therapies in biological systems as an alternative to liposome based systems which are not ideal because they have been associated with cell toxicity [21, 22].

### Hydrophobic Drug Uptake and Release

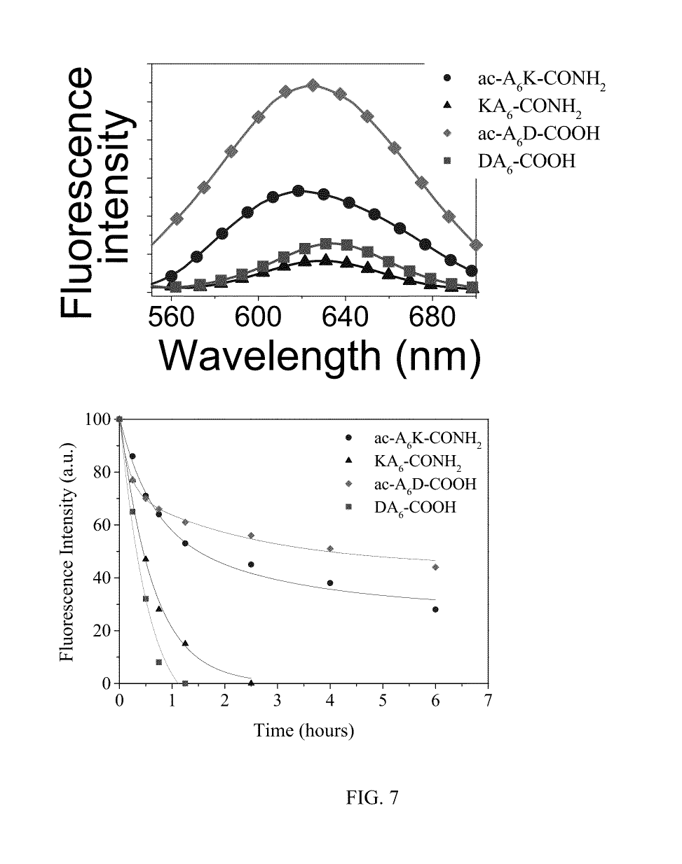

We next studied the hydrophobic character and structural integrity of the peptide vesicle bilayer using Nile red as a probe molecule. Nile red is considered as a hydrophobic model drug and it is used to assess liposome bilayer stability [23]. The fluorescence emitted by Nile red in water is very weak and shows maximum at 660 nm. However, the intensity increases and the maximum is blue shifted when Nile red is buried in a hydrophobic environment where it is shielded from contact with the polar solvent. The emission spectra of Nile red incorporated into the self-assembling peptide vesicles are shown in FIG. 7.

Nile red emission maxima at 623 nm and 626 nm were observed in peptide nanovesicle formulations consisting of the divalent KA₆-CONH₂(SEQ ID NO: 2) and ac-A₆D-COOH (SEQ ID NO: 3), respectively and at 631 nm and 647 nm for the monovalent ac-A₆K-CONH₂ (SEQ ID NO: 1) and DA₆-COOH. (SEQ ID NO: 4) peptide nanovesicles, respectively (FIG. 7). This suggests that Nile red is located in a more hydrophobic microenvironment (i.e., inserted deeper in the peptide bilayer and interacts less with the polar solvent) in nanovesicles of peptides carrying two charges compared to the spatial configuration of Nile red encapsulated in nanovesicles consisting of peptides carrying one charge, regardless of the type of the charge. Nile red encapsulated in ac-A₆KCONH₂ (SEQ ID NO: 1) nanovesicles had fluorescence yield significantly higher compared to those recorded for Nile red in other peptide nanovesicle systems which suggests that larger number of Nile red molecules are accommodated in the ac-A₆K-CONH₂ (SEQ ID NO: 1) nanovesicle bilayer compared to Nile red in other peptide nanovesicles.

The release kinetics of Nile red through peptide nanovesicles was also studied and we found that release was slower in the case of the ac-A₆D-COOH (SEQ ID NO: 3) and ac-A₆K-CONH₂ (SEQ ID NO: 1) nanovesicles and therefore it is suggested that peptide nanovesicles can be used for drug or gene delivery therapies.

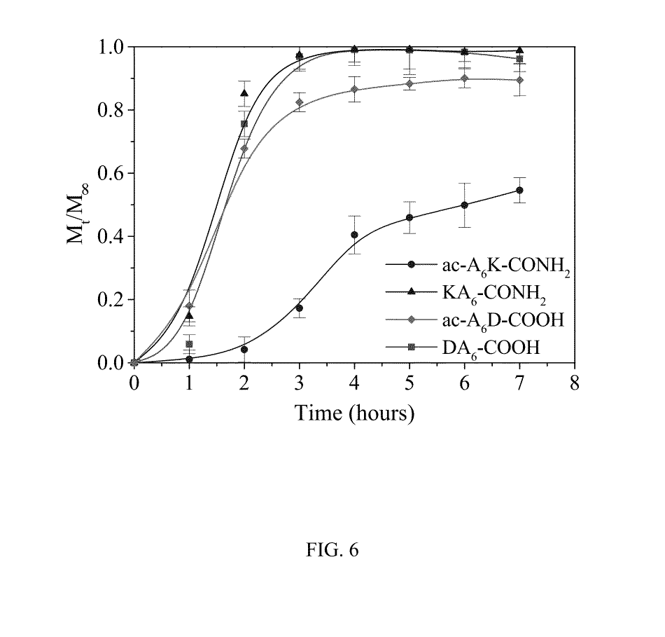

Together, we found that the hydrophobic Nile red molecules are inserted in the bilayer which is composed of the lipid-like peptides' hydrophobic tails that self-assemble to form the vesicle. Furthermore, there seems to be a correlation between membrane bilayer stability of the ac-A₆K-CONH2 (SEQ ID NO: 1) peptide vesicles and their property to retain hydrophobic and hydrophilic molecules (FIGS. 6 and 7). As in the case of CF release, we observed that ac-A₆K-CONH₂ (SEQ ID NO: 1), and to a lesser extent ac-A₆D-COOH (SEQ ID NO: 3) nanovesicles retain higher numbers of Nile red molecules compared to KA₆-CONH₂ (SEQ ID NO: 2) and DA₆-COOH (SEQ ID NO: 4) vesicles,

### Effect of Lipid-Like Peptide Nanovesicles on Cell Viability

The viability of Caco-2 cells in the presence of the four amphiphilic peptides was analyzed and the results suggest that all peptides did not affect cell growth after 24 h and 48 h. FIG. 9 shows that Caco-2 cells exhibit a ˜5-fold cell number increase over a 2-days culture suggesting active proliferation of the cells in the presence of the amphiphilic peptides. To quantitatively assess the effect of self-assembling peptides on Caco-2 cell viability we used the MTT assay with added 0.2 mg/mL and 1.0 mg/mL of peptides. These peptide concentrations are higher than the peptides' CMC values and therefore peptide nanovesicles as well as peptide monomers are present in the system. As shown in FIG. 9, no change in cell viability was observed when cells were incubated with the peptides (t-test p<0.05).

Notably, 48 hours post-treatment in the presence of the acetylated ac-A₆K-CONH₂ (SEQ ID NO: 1) and ac-A₆DCOOH (SEQ ID NO: 3) lipid-like peptides we observed increased cell overgrowth resulting in viability values exceeding 100% (FIG. 9). We anticipated that the addition of self-assembling with surfactant properties would have no beneficial effect on cell growth. This finding was surprising and implies a positive cross-talk between cells and lipid-like peptide monomers or peptide nanovesicles.

### Lipid-Like Peptides as Permeation Enhancers

Having established the lipid-like peptide nanovesicles can be used for drug delivery application through encapsulation of hydrophilic and/or hydrophobic drugs compounds, we set out to explore the ability of peptide nanovesicles to facilitate and amplify the bioavailability of the therapeutic molecules. Successful management of many diseases requires efficient drug delivery. However, delivery of therapeutic agents in effective concentrations is challenging due to tow permeability of the epithelial layers of the gastrointestinal tract or the blood brain barrier. To increase bioavailability various permeation enhancers have been developed. Transwell filter-grown Caco-2 epithelial cells are considered as an in vitro model system to study epithelial permeability [24]. To assess the effect of self-assembling peptides on permeability through Caco-2 cell monolayers we used the high molecular weight hydrophilic marker FITC-dextran MW 4.4 kDa. Paracellular permeability is regulated by the cell's tight junctions which consist of multiple transmembrane proteins and functions as a semi-permeable gate to regulate the flux of luminal fluid and molecules through epithelial layers.

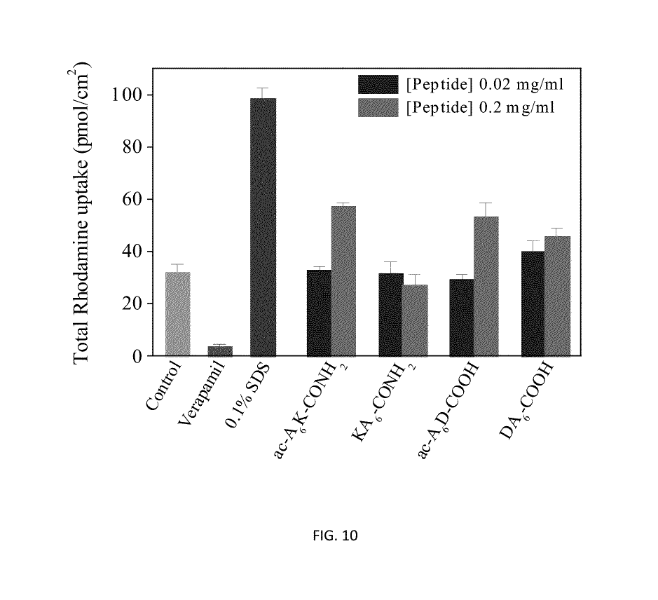

As shown in FIGS. 10 (A & B) the addition of peptides caused a significant increase (p<0.05) in FITC-dextran transport compared to the control (i.e., absence of peptides in apical side of the Transwells). FITC-dextran transport across monolayers without co-application of self-assembling peptides is small, indicating that FITC-dextran alone permeates poorly the Caco-2 mono layers. The presence of 1.0 mgmt ac-A₆D-COOH (SEQ ID NO: 3), DA₆-COOH (SEQ ID NO: 4), ac-A₆K-CONH2 (SEQ ID NO: 1), and KA₆-CONH2 (SEQ ID NO: 2), enhanced the transport of FITC-dextran by 7.9, 6.6, 5.7, and 5.1-fold, respectively compared with the control (FIG. 9 and Table 4). In a similar manner when 0.2 mg/mL, of surfactant-like peptides were used the FITC-dextran transport was enhanced following the same order of efficiency as previously observed for tests performed at 1.0 mgmt of added peptide.

Permeation enhancement ratios in this range may result in increased drug transport through the epithelial layer without risk of compromising the epithelial layer integrity. Although the Caco-2 cell model does not include the mucus layer, which is present in some epithelial layers, high absorption enhancement ratios in vitro are associated with improved pharmacokinetics. These data show that there is consistently increased permeation of HTC-dextran through the epithelial monolayer at higher peptide concentrations and that the effect is more pronounced in the case of negatively charged peptides nanovesicles.

To investigate the effect of the surfactant-like peptides on the tight junction barrier, we measured the transepithelial resistance across the Caco-2 monolayer (FIGS. 9 C & D). Reduction of the transepithelial resistance (an index of paracellular permeability) across the epithelial monolayer indicates increased paracellular permeability. The observed, time-dependent, small decrease in the transepithelial resistance is due to increased permeability of the monolayer to small ions in the presence of 10 mil HBSS/HEPES buffer pH 7.4 as a result of dilatation of the tight junctions between the cells. Our results show that the application of the surfactant-like peptides did not significantly affect the transepithelial resistance of the monolayer which was comparable to the control. This result is in agreement with the cell viability data (FIG. 8) which show that after 180 min the cell population was not affected by the presence of the peptides. Together these data suggest that lipid-like peptides interact with the tight junctions of the epithelial monolayer and increase the permeability of FITC-dextran without compromising the Caco-2 cell monolayer integrity. A comparison of the results presented in FIG. 9 also suggest that the presence of the smaller nanovesicles, derived from self-association of aspartic-containing lipid-like peptides, result in increased FITC-dextran transport and TEER values which are closer to that obtained for the control compared to the values obtained for ac-A₆K-CONH₂ (SEQ NO: 1) and KA₆-CONH₂ (SEQ ID NO: 2) nanovesicles.

At the end of the TEER experiment (i.e., 180 min, arrow in FIGS. 9 C & D) the cells were supplied with peptide-depleted growth medium and the transepithelial resistance was measured up to 300 min. In all cases, we found that the transepithelial resistance returned to 95-100% of the pre-treatment values suggesting that the observed TEER changes are reversible and cannot due to damaging the tight junctions or to adverse impairment of the cell membrane function.

Rhodamine-123 Transport Through Caco-2 Monolayers in the Presence of Peptide nanovesicles

Having observed that self-assembling peptides interact with epithelial cell monolayers and increase paracellular permeability and FITC-dextran transport we set out to explore the mechanism of permeability by testing the P-glycoprotein mediated efflux. P-glycoprotein is localized mainly in the apical surface of the epithelial layer, including the human brain capillary blood vessels that are responsible for the blood brain barrier, and it is associated with the active efflux of many drugs [25]. Specific surfactants that are included in pharmaceutical formulations to improve the drug solubility also interact with the P-glycoprotein and thus affect the pharmacokinetic parameters [26, 27].

An ideal drug delivery vehicle should be able to modulate the P-glycoprotein function and increase drug bioavailability. To this end, we sought to evaluate the ability of amphiphilic peptide formulations to affect P-glycoprotein function in Caco-2 cell monolayers. Transport of Rhodamine-123, which is a substrate for P-glycoprotein [28], was measured through the epithelial monolayer with and without added surfactant-like peptides. Verapamil, which is an inhibitor of the P-glycoprotein cellular function, was included in our assay as control [29]. We performed measurements for the transport (from the basolateral to the apical side) because the influx across the basal membrane (absorptive transport) is not controlled by the P-glycoprotein function [28].

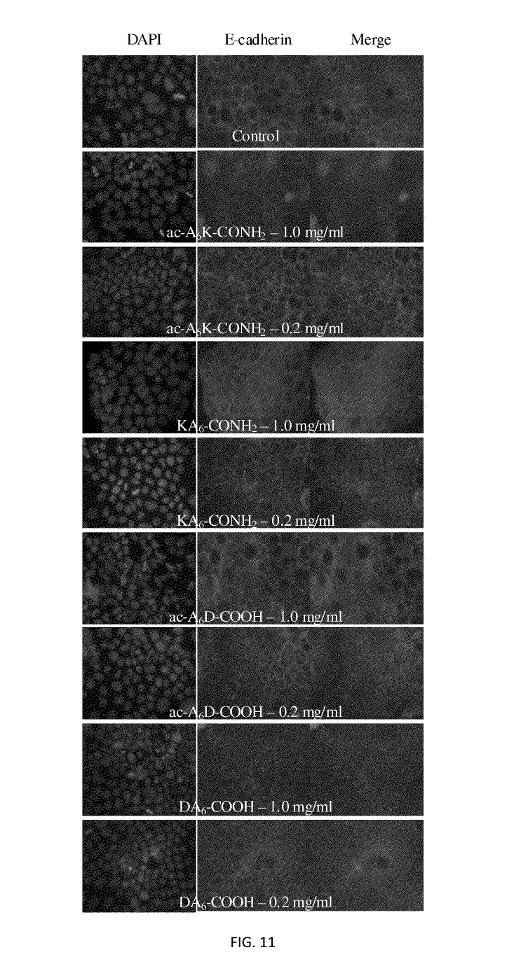

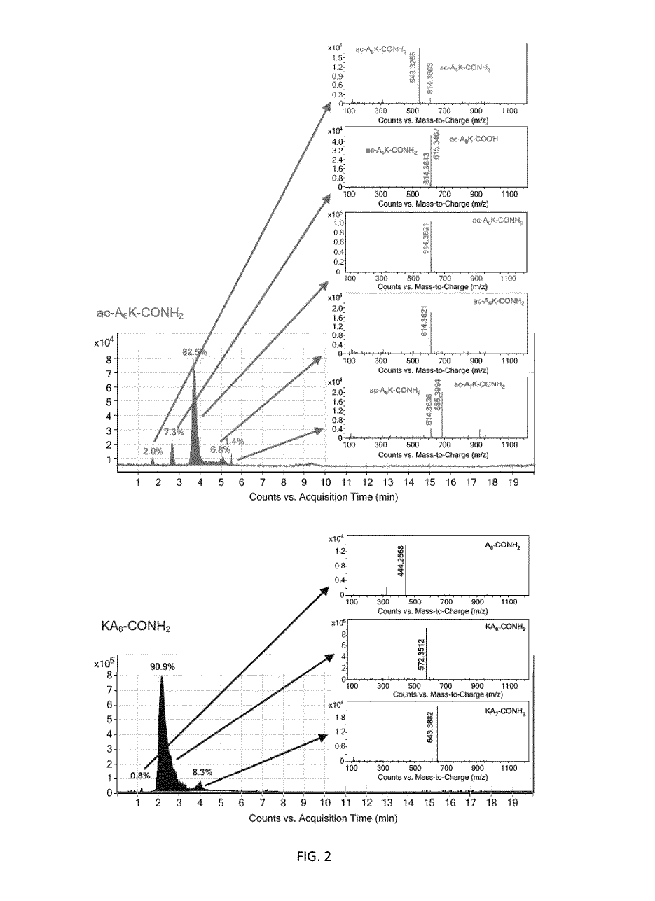

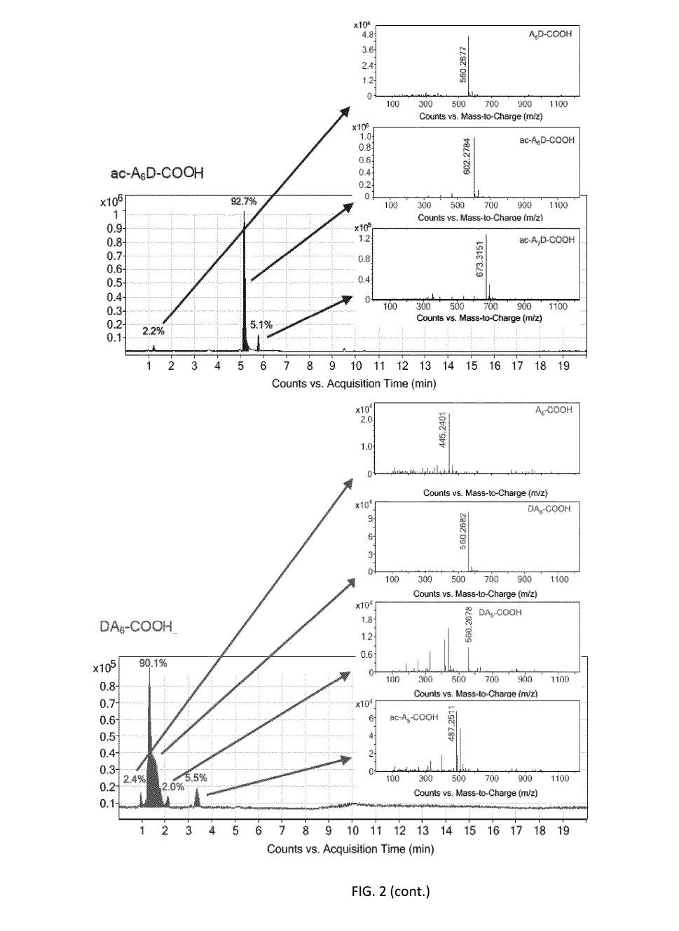

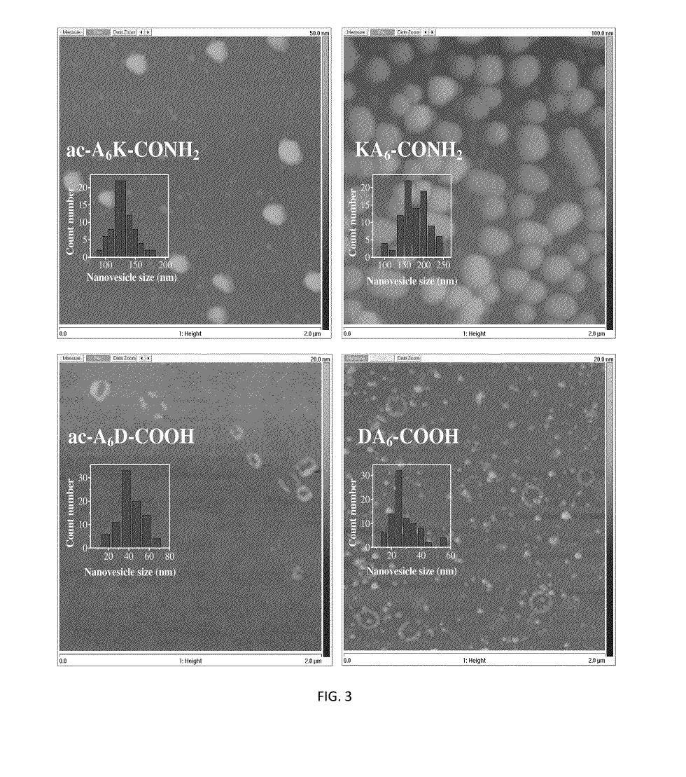

Verapamil inhibited the transport of Rhodamine-123. The presence of self-assembling peptide monomers at concentrations below their CMC did not induce changes in the Rhodamine-123 transport compared to the control (FIG. 11). However, addition of ac-A₆K-CONH₂ (SEQ ID NO: 1) and ac-A₆D-COOH (SEQ ID NO: 3) peptide nanovesicles viz, at concentrations above their CMC, resulted in Rhodamine-123 transport values through the cell monolayer that were consistently higher compared to the control. The presence of KA₆CONH₂ (SEQ ID NO: 2) and DA₆-COOH (SEQ ID NO: 4) nanovesicles did not affect the Rhodamine-123 transport.

These data demonstrate that ac-A₆K-CONH₂ (SEQ ID NO: 1) and ac-A₆D-COOH (SEQ ID NO: 3) peptide nanovesicles facilitate the secretory transport of Rhodamine-123 possible through a mechanism that involves interactions between peptide nanovesicles and P-glycoprotein. To further test this hypothesis and to investigate the biological effect of peptide nanovesicles on cellular morphology and adhesion to epithelial cell tight junctions we performed immunostaining using a monoclonal antibody against the epithelial membrane antigen E-cadherin. E-cadherin is a transmembrane glycoprotein that is localized in the adherens junctions of epithelial cells and regulates epithelial tight junction formation. Down-regulation or redistribution of tight and adherens junction complexes away from the apicolateral membrane is correlated with disruption of the cell monolayer allowing free diffusion of small molecules, proteins and lipids through the membrane between apical and basolateral domains:

Our results show that surfactant-like peptides at concentrations below and above their CMC do not affect the Caco-2 cell morphology or adversely modify the tight junctions integrity. E-cadherin staining is strongly positive in peptide-treated Caco-2 cell membranes and fluorescence microscopy did not reveal differences between negative control and peptide treated monolayers. This suggests that the increased permeability of FITC-dextran MW 4.4 kDa and Rhodamine-123 transport through the epithelial monolayer is not due to cell membrane compromise. It is likely that the acetylated lipid-like peptides acA₆K-CONH₂ (SEQ ID NO: 1) and ac-A₆D-COOH (SEQ ID NO: 3) enhance permeation by a mechanism that involves paracellular and/or intracellular transport of the diffusant. Together our results demonstrate that lipid-like peptides monomers and peptide nanovesicles do not affect cell morphology, cell viability, and proliferation properties suggesting that peptide nanovesicles are potential good candidates for biomedical applications including drug delivery,

### Features of the Peptide Nanovesicles

All peptides were designed with a hydrophobic tail and a hydrophilic head which self-assemble to form nanovesides. Our results show that the peptide's surfactant properties alone and the formation of nanovesicles is a necessary but not sufficient condition for the development of an efficient drug delivery system. Although all peptide vesicles maintain their macroscopic organization some of them fail to retain the encapsulated molecules for prolonged times. We observed encapsulation of the hydrophobic probe Nile red in the vesicle bilayer and prolonged release of the hydrophilic CF through the ac-A₆K-CONH₂ (SEQ ID NO: 1) peptide nanovesicles with size ˜126 nm. Self-assembly of ac-A6D-COOH (SEQ ID NO: 3) results in small nanovesicles of ˜43 nm which efficiently encapsulate the hydrophobic Nile red in the peptide bilayer but release the negatively charged CF faster compared to ac-A₆K-CONH₂ (SEQ ID NO: 1) peptide vesicles. These results suggest that the size of the nanovesicles and their loading capacity provide a means to control the release kinetics of a drug by altering the charge of the self-assembling peptide monomers that are used to form vesicles.

The amino acid sequence, which defines the position of the charges on the peptide, is also important for designing an efficient system. While the ac-A₆K-CONH₂ (SEQ ID NO: 1) nanovesicles significantly retained the negatively charged CF, the KA₆-CONH₂ (SEQ ID NO: 2) nanovesicles did not, although KA₆-CONH₂ (SEQ ID NO: 2) carries two positive charges per monomer (at pH 7.4, the non-acetylated N-terminal amine and the c-amine of lysine are charged, Table 1). Fast CF release was also observed through DA₆-COOH (SEQ ID NO: 4) nanovesicles compared to ac-A₆D-COOH (SEQ ID NO: 3) formulations; DA₆-COOH (SEQ ID NO: 4) carries one negative charge at the C-terminal and it is neutral at the N-terminal (one positive and one negative) whereas ac-A₆D-COOH (SEQ ID NO: 3) carries two negative charges. Hence, the charge distribution is also an important factor for the design of efficient, peptide nanovesicle drug delivery systems.

For the formation of stable vesicles a good amphiphilic peptide should have (i) acetylated N terminus like ac-A₆K-CONH₂ (SEQ ID NO: 1) and ac-A₆D-COOH (SEQ ID NO: 3); the non-acetylated peptides KA₆-CONH, (SEQ ID NO: 3) and DA₆-COOH (SEQ ID NO: 4) did not retain CF, (ii) positively charged amino acid in the C-terminus; lysine at the N-terminus did not result in stable peptide nanovesicles with good CF retention properties, (iii) amidated C-terminus; the ac-A₆D-COON peptide with free carboxyl group at the C-terminus showed only a small CF retention efficiency.

Although the qualitative process of self-assembly of peptides is similar to that of lipids and fatty acids, peptides differ from these systems because the nanovesicle bilayer is stabilized by a combination of hydrophobic interactions between the hydrophobic side groups of the amino acids and hydrogen bonding between the peptide monomer polar backbones. Therefore, the bilayer internal chemistry differs between liposomes and peptide nanovesicles. We believe that these simple, short, inexpensive (less than $45 per gram) and versatile peptides will open new paths in the field of vesicle-assisted drug delivery applications. Surfactant-like, self-assembling peptides may be mixed with lipids to form hybrid peptidelipid liposome systems for targeted drug delivery. The incorporation of self-assembling peptides in liposomes conferred functionality and modulated the bilayer curvature and the stability of the formulation [30]. Surfactant-like peptides can be easily modified and tailored to incorporate other molecules such as sugars and functional motifs, including cell signaling and cell penetrating amino acid sequences. Engineering of designer peptides is an enabling technology that will likely play an increasingly important role in the coming decades for their use for biomedical applications.

TABLE 1
Amino acid sequence, charge distribution, and physicochemical properties of the
amphiphilic, self-assembling peptide suspensions. Indicated domains represent amino
acids with positive charge (underlined), negative charge (italicized), and hydro-
phobic side chains. The charge of the peptides at pH 7.4 was calculated using pKa
values from the literature.

| | Peptide Sequence | Net charge | CMC in |
|---|---|---|---|
| Peptide | and charge distribution | at pH 7.4 | PBS (mg/ml) |
| Ac-A₆K-CONH2 | Acetyl-Ala-Ala-Ala-Ala-Ala-Ala-Ala-Lys- | +1 | 0.16 |
| (SEQ ID NO: 1) | CONH2 | | |
| KA₆-CONH2 | +NH3-Lys-Ala-Ala-Ala-Ala-Ala-Ala-Ala- | +2 | 0.07 |
| (SEQ ID NO: 2) | CONH2 | | |
| Ac-A₆D-COOH | Acetyl-Ala-Ala-Ala-Ala-Ala-Ala-Ala-Asp- | -2 | 0.05 |
| (SEQ ID NO: 3) | COO- | | |
| DA₆-COOH | +NH3-Asp-Ala-Ala-Ala-Ala-Ala-Ala-Ala- | -1 | 0.12 |
| (SEQ ID NO: 4) | COO- | | |

TABLE 2
Size of individual lipid-like peptide nanovesicles and peptide nanovesicle beads
and clusters in the dry state as determined by AFM image processing and DLS curve
fitting of peptide nanovesicles in PBS solution.

| | Vesicle diameter | Vesicle diameter | Clusters | Beads diameter |
|---|---|---|---|---|
| | (nm) | (nm) | diameter (nm) | (nm) |
| Peptide | AFM analysis | DLS analysis | AFM analysis | AFM analysis |
| Ac-A₆K-CONH2 | 126 ± 23 | 122 (30-173) | - | - |
| (SEQ ID NO: 1) | | | | |
| KA₆-CONH2 | 169 ± 29 | 164 (65-350) | - | - |
| (SEQ ID NO: 2) | | | | |
| Ac-A₆D-COOH | 28 ± 9 | 97 (30-320) | 101 ± 11 | 200 ± 11 |
| (SEQ ID NO: 3) | | | | |
| DA₆-COOH | 43 ± 11 | 137 (64-725) | 135 ± 24 | 159 ± 26 |
| (SEQ ID NO: 4) | | | | |

TABLE 3
Permeability parameters of FITC-dextran 4.4 kDa through
a Caco-2 monolayer in the presence of 0.2 mg/mL and 1.0
mg/mL of lipid-like a peptides.

| | Concentration | Pₐₚₚ | Enhancement |
|---|---|---|---|
| Peptide | (mg/ml) | (10⁻⁵ cm/s) | ratio |
| Control | - | 0.62 ± 0.33 | - |
| Ac-A₆K-CONH2 | 0.2 | 1.20 ± 0.36 | 4.4 |
| (SEQ ID NO: 1) | 1.0 | 1.51 ± 0.37 | 5.7 |
| KA₆-CONH2 | 0.2 | 1.08 ± 0.30 | 4.0 |
| (SEQ ID NO: 2) | 1.0 | 1.38 ± 0.22 | 5.1 |
| Ac-A₆D-COOH | 0.2 | 1.82 ± 0.24 | 6.7 |
| (SEQ ID NO: 3) | 1.0 | 2.13 ± 0.16 | 7.9 |
| DA₆-COOH | 0.2 | 1.37 ± 0.38 | 5.0 |
| (SEQ ID NO: 4) | 1.01 | 1.78 ± 0.17 | 6.6 |

TABLE 4
Permeability parameters of Rhodamine-123 through a
Caco-2 monolayer (from the basolateral to the apical
side) in the presence of the P-glycoprotein inhibitor
Verapamil, SDS 1%, and 0.02 mg/mL and 0.2 mg/mL of
lipid-like peptides.

| | Concentration | Pₐₚₚ | Enhancement |
|---|---|---|---|
| Peptide | (mg/ml) | (10⁻⁵ cm/s) | ratio |
| Control | - | 1.10 ± 0.13 | - |
| Verapamil | - | 0.13 ± 0.03 | 0.1 |
| SDS 1% | - | 3.28 ± 0.02 | 3.0 |
| Ac-A₆K-CONH₂ | 0.02 | 1.00 ± 0.05 | 0.9 |
| (SEQ ID NO: 1) | 2 | 1.82 ± 0.04 | 1.7 |
| KA₆-CONH₂ | 0.02 | 1.08 ± 0.02 | 1.0 |
| (SEQ ID NO: 2) | 2 | 1.05 ± 0.02 | 1.0 |
| Ac-A₆D-COOH | 0.02 | 1.01 ± 0.06 | 0.9 |
| (SEQ ID NO: 3) | 2 | 1.84 ± 0.02 | 1.7 |
| DA₆-COOH | 0.02 | 1.30 ± 0.02 | 1.2 |
| (SEQ ID NO: 4) | 2 | 1.51 ± 0.01 | 1.4 |

### CONCLUSIONS

Inspired by biological lipids, we devised amphiphilic, surfactant-like, peptides with self-assembling properties which form nanovesicles that can be used for drug delivery applications of hydrophobic and hydrophilic molecules. We investigated the physicochemical and morphological properties of peptide vesicles in solution and we observed good in vitro biocompatibility of the peptide formulations. We demonstrated that peptide nanovesicles facilitate transport through epithelial monolayers via interaction with the P-glycoprotein system. Cell proliferation studies showed that amphiphilic peptides did not affect cell growth in Caco-2 cell cultures. Self-assembling peptide vesicles represent a new type of nanomaterial with potential utilities in biomedicine. These amphiphilic peptides not only can be readily designed at the single amino acid level but also are easily made through standard peptide synthesis. Our results provide further understanding towards the development of a peptide-based drug delivery system in which molecules with therapeutic properties will be encapsulated and slowly delivered in the body.

The Center for Environmental Health Sciences (CEHS; Grant No. N1EHS ES0021 09) at MIT is acknowledged for mass spectrometric analysis and technical support.

### REFERENCES

1. Whitesides, G. M., Mathias, J. P. & Seto, C. T. (1991) Science, 254, 1312-1319.
2. Georger, J. H., Singh, A., Price, R. R., Yager, P. & Schoen, P. E. (1987) J. Am. Chem. Soc., 109, 6169-6175,
3. Holmes, T. C., De Lecalle, S., Su, X., Liu, G., Rich, A. & Zhang, S. (2000) Proc. Nat/. Acad. Sci. USA 97, 6728-6733.
4. Koutsopoulos, S., Unsworth, L. D., Nagai, Y. & Zhang, S. G. (2009) Proc. Nat/. Acad. Sci. USA, 106, 4623-4628.
5. Nagai, Y., Unsworth, L. D., Koutsopoulos, S. & Zhang, S. (2006) J. Control. Rei., 115, 18-25.
6. Vauthey, S., Santoso, S., Gong, H., Watson, N. & Zhang, S. (2002) Proc. Nat. Acad. Sci. USA, 99, 5355-5360.
7. Santoso, S., Hwang, W., Hartman, H. & Zhang, S. (2002) Nano Lett., 2, 687-691.
8. Nagai, A., Nagai, Y., Qu, H. & Zhang, S. (2007) J. Nanosci. Nanotechnol., 7, 1-7.
9. Khoe U., Yang Y. L. & Zhang S. G. (2009) Langmuir, 25, 4111-4114.
10. Matsumoto, K., Vaughn, M., Bruce, B. D., Koutsopoulos, S. & Zhang, S. (2009) J. Phys. Chern. B, 113, 75-83.
11. Wang, X. Q., Carin, K., Baaske, P., Wienken, C. J., Jerabek-Willemsen, M., Duhr, S., Braun, D. & Zhang, S. G. (2011) Proc. Nat Acad, Sci. USA, 108, 9049.
12. Das, R., Kiley, P. J., Segal, M., Norville, J., Yu, A. A., Wang, L. Y., Trammell, S. A., Reddick, L. E., Kumar, R., Stellacci, F., Lebedev, N., Schnur, J., Bruce, B. D., Zhang, S. & Baldo, M. (2004) Nano Lett., 4, 1079-1083.
13. Berne, B. J. & Pecora, R. (1976) Dynamic light scattering, Wiley, New York.
14. Provencher, S. W. (1982) Comp. Phys. Comm., 27, 213-227.
15. Iqbal, T., Kinjo, M, & Dowling, T. C. (2005). J. Chromatogr. BAnal. Techno/. Biomed. Life Sci., 814, 259-262.
16. Lasic, D. D. (1995). J. Liposome Res., 5, 431-441,
17. Denkov, N O, Velev, 00, Kralchevsky, P A, Ivanov, I B, Yoshimura, H, Nagayama, K. (1993) Twodimensional crystallization. Nature, 361, 26-26.
18. Deegan, R D., Bakajin, 0., Dupont, T F., Huber, G., Nagel, S R. & Witten, T A (1997) Capillary flow as the cause of ring stains from dried liquid drops. Nature, 389, 827-829.
19. Allen, M. J., Hud, N. V., Balooch, M., Tench, R. J., Siekhaus, W. J. & Balhom, R. (1992) Ultramicroscopy, 42, 1095-1100.
20. Florence, A. T. & Attwood, D. (1998) Physicochemical Principles of Pharmacy. 3rd Edition, Macmillan Press Ltd., Chapter 7.
21. Zhu, N., Liggitt, D., Liu, Y. & Debs, R. (1993) Science, 261, 209-211.
22. Filion, M. C. & Phillips, N. C. (1997) Biochim. Biophys. Acta—Biomembranes, 1329, 345-356.
23. Greenspan, P. & Fowler, S. D. (1985) J. lip. Res., 26, 781-789.
24. Hidalgo, U; Raub, T J; Borchardt, R T (1989) Characterization of the human-colon carcinoma cell-line (Caco-2) as a model system for intestinal epithelial permeability. Gastroenterology 96, 736-749.
25. Cordon-Cardo, C., O'Brien, J. P., Boccia, J., Casals, D., Bertino, J. R. & Melamed, M. R. (1990) J. Histochem. Cytochein. 38, 1277-1287.
26. Bagman, K., Erne-Brand, F., Alsenz, J. & Drewe, J. (2003) J. Pharm., Sci., 92, 1250-61.
27. Tayrouz, Y., Ding, R., Burhenne, J., Riedel, K. D., Weiss, J., Hoppe-Tichy, T., Haefeli, W. E. & Mikus, G (2003) Clin. Pharmacal. Ther. 73, 397-405.
28. Troutman, M. D. & Thakker, D. R. (2003) Pharm. Res. 20, 1192-1199.
29. Lehnert, M., Dalton, W. S., Roe, D., Emerson, S. & Salmon, S. E. (1991) Blood, 77, 348-354.
30. Yaghmur, A., Laggner, P., Zhang, S. & Rappolt, M. (2007) PLoS ONE, 5, 1-10.

While this invention has been particularly shown and described with references to preferred embodiments thereof, it will be understood by those skilled in the art that various changes in form and details may be made therein without departing from the scope of the invention encompassed by the appended claims.

1. A drug delivery composition comprising a self-assembled nanostructure and a biologically active molecule, wherein the nanostructure comprises a plurality of surfactant peptides and wherein the surfactant peptides have a formula selected from the group consisting of:
a. (Φ)ₘ(+)ₙ (Formula (1)),
b. (+)ₙ(Φ)ₘ (Formula (2)),
c. (Φ)ₘ(−)ₙ (Formula (3)),
d. (−)ₙ(Φ)ₘ (Formula (4)),
e. (−)ₙ(Φ)ₘ(−)ₙ (Formula (5)),
f. (+)ₙ(Φ)ₘ(+)ₙ (Formula (6)),
g. (Φ)ₘ(−)ₙ(Φ)ₘ (Formula (7)),
h. (Φ)ₘ(+)ₙ(Φ)ₘ (Formula (8)),
i. (+)ₙ(Φ)ₘ(−)ₙ (Formula (9)), and
j. (−)ₙ(Φ)ₘ(+) (Formula (10)),
wherein:
(Φ) represents independently for each occurrence, a natural or non-natural amino acid comprising a hydrophobic side-chain;
(+) represents independently for each occurrence a natural or non-natural amino acid comprising a side-chain that is cationic at physiological pH;
(−) represents independently for each occurrence a natural or non-natural amino acid comprising a side-chain that is anionic at physiological pH;
wherein the terminal amino acids are optionally substituted;
m for each occurrence represents an integer greater than or equal to 5; and
n for each occurrence represents an integer greater than or equal to 1;
and further wherein the biologically active agent is encapsulated within the nanostructure.

2. The composition of claim 1, wherein the surfactant peptide has the Formula (1) or the Formula (3).

3. The composition of claim 2, wherein (Φ) is selected from the group consisting of alanine, valine, leucine, isoleucine and proline.

4. The composition of claim 3, wherein (Φ) is alanine.

5. The composition of claim 3, wherein (+) is selected from the group consisting of histidine, lysine or arginine.

6. The composition of claim 3, wherein (−) is selected from the group consisting of aspartic acid or glutamic acid.

7. The composition of claim 1, wherein the N-terminal amino acid of the surfactant peptides is substituted by an acetyl group.

8. The composition of claim 1, wherein the C-terminal amino acid of the surfactant peptides is substituted with an amino group.

9. The composition of claim 1, wherein n is 1.

10. The composition of claim 1, wherein m is 5, 6 or 7.

11. The composition of claim 9, wherein m is 6.

12. The composition of claim 1, wherein the surfactant peptide is selected from the group consisting of ac-AAAAAAK-CONH₂ (SEQ ID NO: 1), KAAAAAA (SEQ ID NO: 2), ac-AAAAAAD-COOH (SEQ ID NO: 3) and DAAAAAA-COOH (SEQ ID NO: 4).

13. The composition of claim 12, wherein the surfactant peptide is selected from the group consisting of ac-AAAAAAK-CONH₂ (SEQ ID NO: 1) and ac-AAAAAAD-COOH (SEQ ID NO: 3).

14. The composition of claim 1, wherein the nanostructure is a nanovesicle.

15. The composition of claim 1, wherein the biologically active molecule is hydrophobic.

16. The composition of claim 1, wherein the biologically active molecule is hydrophilic.

17. The composition of claim 1, wherein the biologically active molecule is a nucleic acid.

18. The composition of claim 1, wherein the surfactant peptides have an acetylated N-terminus.

19. The composition of claim 1, wherein the surfactant peptides have a positively charged amino acid at the C-terminus.

20. The composition of claim 1, wherein the surfactant peptides have an amidated C-terminus.

21. The composition of claim 1, further comprising a liposome.

22. A method for administering a biologically active molecule to a subject comprising administering to said subject a composition of claim 1.

23. The method of claim 22, wherein the biologically active molecule is delivered to the gastrointestinal tract or across the blood brain barrier of said subject.

24. The method of claim 23, wherein the biologically active molecule is delivered to the gastrointestinal tract.

25. The method of claim 24, wherein the surfactant peptides are negatively charged.

26. The method of claim 1, wherein the biologically active molecule in the composition has a controlled release profile.

27. A method of preparing the composition of claim 1, comprising bringing into contact a biologically active molecule with the surfactant peptides in an aqueous solution under conditions suitable for self-assembly.


SEQUENCE LISTING (4 sequences)

SEQ ID NO: 1 - protein, 7 aa, Artificial Sequence
  Synthetic
AAAAAAK

SEQ ID NO: 2 - protein, 7 aa, Artificial Sequence
  Synthetic
KAAAAAA

SEQ ID NO: 3 - protein, 7 aa, Artificial Sequence
  Synthetic
AAAAAAD

SEQ ID NO: 4 - protein, 7 aa, Artificial Sequence
  Synthetic Peptide
DAAAAAA